(19) United States
(12) Patent Application Publication
(10) Pub. No.: US 2024/0366908 A1
(43) Pub. Date: Nov. 7, 2024

(54) TIME OR TIDAL VOLUME SPLITTING VENTILATOR AND METHODS OF USE

(71) Applicants: H. LEE MOFFITT CANCER CENTER AND RESEARCH INSTITUTE, INC., Tampa, FL (US); UNIVERSITY OF SOUTH FLORIDA, Tampa, FL (US)

(72) Inventors: Aaron MUNCEY, Tampa, FL (US); Heiko ENDERLING, Tampa, FL (US); Stefano PASETTO, Tampa, FL (US); Christopher PASSAGLIA, Lutz, FL (US); Jacob YARINSKY, Tampa, FL (US); Carolyna Yamamoto Alves PINTO, Baltimore, MD (US); Abby BLOCKER, Inverness, FL (US)

(21) Appl. No.: 18/555,600

(22) PCT Filed: Apr. 18, 2022
(86) PCT No.: PCT/US2022/025218

§ 371 Date: Oct. 16, 2023

Related U.S. Application Data
(60) Provisional application No. 63/176,039, filed on Apr. 16, 2021.
(60) Provisional application No. 63/320,453, filed on Mar. 16, 2022.

(51) Int. Cl. A61M 16/20; A61M 16/08

(52) U.S. Cl. CPC A61M 16/204; A61M 16/0833; A61M 16/0875; A61M 2205/3334; A61M 2205/505; A61M 2205/84

(57) ABSTRACT

A ventilator device for co-ventilation of multiple patients may include an air tube splitter having an inlet, a plurality of outlets, and a plurality of branches each extending to a respective outlet, a plurality of valves coupled to the branches, a plurality of actuators coupled to the valves, and a controller in operable communication with the actuators. Each valve may be configured to be adjusted to regulate air flow through a respective branch. Each actuator may be configured to adjust a respective valve. The controller may be configured to receive a set of inspiratory pressure settings or tidal volume settings for the patients and independently control the actuators to adjust the valves based at least in part on the set of inspiratory pressure settings or tidal volume settings. The controller may employ time-multiplexing in controlling the actuators to adjust the valves for ventilating the patients at different times.

### CROSS-REFERENCE TO RELATED APPLICATIONS

This application claims the benefit of, and priority to, U.S. Provisional Patent Application No. 63/320,453, filed on Mar. 16, 2022, and titled “Time or Tidal Volume Splitting Ventilator and methods of Use,” and U.S. Provisional Patent Application No. 63/176,039, filed on Apr. 16, 2021, and titled “Time or Tidal Volume Splitting Ventilator and methods of Use,” the disclosures of which are expressly incorporated herein by reference in their entirety.

### FIELD OF THE DISCLOSURE

The present disclosure generally relates to ventilators and, more particularly, to ventilator devices and methods of using such devices for co-venting multiple patients.

### BACKGROUND OF THE DISCLOSURE

The use of a single ventilator among a plurality of patients has been implemented to provide ventilation for patients when the demand for ventilation exceeds the supply of ventilator machines. Current solutions include inserting a simple splitter in inspiratory ventilation tubing from a central location, with individual tubing segments going to each patient. However, this technique has an inherent problem; the air flow delivery to the patients is only equivalent when the patients' lung compliance and lung size are the same. This is often not the case. In situations where patients have differing lung compliance and size, patients with more compliant lungs receive more air. Other current solutions use valves to regulate air flow to multiple patients, but such valves are not adjustable in real-time, making it difficult to keep the patients properly ventilated as their conditions change. New methods and devices are needed for ventilating multiple patients in a manner such that air flow can be adjusted in real-time to account for patient differences and provide sufficient air to the patients as they breathe from a single ventilator over time.

### SUMMARY OF THE DISCLOSURE

The present disclosure provides ventilator devices, systems, and methods for co-ventilation of multiple patients. In one aspect, a ventilator device for co-ventilation of multiple patients is provided. In one embodiment, the ventilator device may include an air tube splitter having an inlet, a plurality of outlets, and a plurality of branches, with each of the branches extending to a respective one of the outlets. The ventilator device also may include a plurality of valves coupled to the branches, a plurality of actuators coupled to the valves, and a controller in operable communication with the actuators. Each of the valves may be configured to be adjusted to regulate air flow through a respective one of the branches. Each of the actuators may be configured to adjust a respective one of the valves. The controller may be configured to receive a set of inspiratory pressure settings or tidal volume settings for the patients, and independently control the actuators to adjust the valves based at least in part on the set of inspiratory pressure settings or tidal volume settings.

In some embodiments, the plurality of outlets may consist of two outlets, the plurality of branches may consist of two branches, the plurality of valves may consist of two valves, and the plurality of actuators may consist of two actuators. In some embodiments, the plurality of outlets may include three or more outlets, the plurality of branches may include three or more branches, the plurality of valves may include three or more valves, and the plurality of actuators may include three or more actuators. In some embodiments, the plurality of branches may include a first branch and a second branch disposed adjacent to one another and defining an angle therebetween. In some embodiments, the angle may be fixed. In some embodiments, the angle may be between 15 degrees and 180 degrees. In some embodiments, the angle may be adjustable. In some embodiments, the angle may be adjustable between 15 degrees and 180 degrees. In some embodiments, the air tube splitter may include an adjustable connection disposed between the first branch and the second branch and configured to allow adjustment of the angle. In some embodiments, the adjustable connection may include a hinge.

In some embodiments, each of the valves may be configured to be adjusted between an open position and a closed position. In some embodiments, the controller may be further configured to control the actuators to alternately adjust the valves between the open position and the closed position based at least in part on a predetermined inspiratory to expiratory ratio pattern. In some embodiments, the controller may be further configured to control the actuators to alternately adjust the valves between the open position and the closed position based at least in part on a predetermined inspiratory to expiratory ratio. In some embodiments, the predetermined inspiratory to expiratory ratio may be 1:1, 1:1.5, 1:2, 1:3, 1:4, or 1:5. In some embodiments, each of the valves may be further configured to be adjusted to a plurality of partially open positions between the open position and the closed position. In some embodiments, the controller may be further configured to control the actuators to adjust the valves such that: a first valve and a second valve of the plurality of valves are in the closed position for a first time period; the first valve is in the open position or a partially open position while the second valve is in the closed position for a second time period following the first time period; the second valve is in the open position or a partially open position while the first valve is in the closed position for a third time period following the second time period; and the first valve and the second valve are in the closed position for a fourth time period following the third time period. In some embodiments, each of the valves may include a ball valve. In some embodiments, each of the valves may include a pinch valve.

In some embodiments, the ventilator device also may include a plurality of first connectors coupled to the branches and the valves, with each of the first connectors connecting a respective one of the valves to a respective one of the branches. In some embodiments, the ventilator device also may include a plurality of second connectors coupled to the valves, with each of the second connectors being configured to connect a ventilation tubing line to a respective one of the valves. In some embodiments, the air tube splitter, the first connectors, the valves, and the second connectors may be separately formed and connected to one another. In some embodiments, the air tube splitter, the first connectors, portions of the valves, and the second connectors may be integrally formed with one another. In some embodiments, each of the actuators may include a motor. In some embodiments, each of the actuators may include a servo motor.

In some embodiments, the ventilator device also may include a plurality of flow sensors coupled to the branches downstream from the valves and in operable communication with the controller, with each of the flow sensors being configured to detect a flow rate of air flow downstream from a respective one of the valves, and with the controller being further configured to receive flow rate signals indicative of flow rates detected by the flow sensors, and independently control the actuators to adjust the valves based at least in part on the flow rate signals. In some embodiments, the ventilator device also may include a plurality of pressure sensors coupled to the branches downstream from the valves and in operable communication with the controller, with each of the pressure sensors being configured to detect a pressure of air flow downstream from a respective one of the valves, and with the controller being further configured to receive pressure signals indicative of pressures detected by the pressure sensors, and independently control the actuators to adjust the valves based at least in part on the pressure signals. In some embodiments, the ventilator device also may include a plurality of carbon dioxide sensors coupled to the branches downstream from the valves and in operable communication with the controller, with each of the carbon dioxide sensors being configured to detect a carbon dioxide concentration of air flow downstream from a respective one of the valves, and with the controller being further configured to receive carbon dioxide concentration signals indicative of carbon dioxide concentrations detected by the carbon dioxide sensors, and independently control the actuators to adjust the valves based at least in part on the carbon dioxide concentration signals. In some embodiments, the ventilator device also may include a plurality of oxygen sensors coupled to the branches downstream from the valves and in operable communication with the controller, with each of the oxygen sensors being configured to detect an oxygen concentration of air flow downstream from a respective one of the valves, and with the controller being further configured to receive oxygen concentration signals indicative of oxygen concentrations detected by the oxygen sensors, and independently control the actuators to adjust the valves based at least in part on the oxygen concentration signals.

In some embodiments, the ventilator device also may include a user interface in operable communication with the controller, with the user interface being configured to allow a user to input and adjust the set of inspiratory pressure settings or tidal volume settings for the patients. In some embodiments, the user interface may be further configured to allow the user to input and adjust an inspiratory to expiratory ratio for the patients. In some embodiments, the user interface may include a display screen, a rotary encoder, and a knob coupled to the rotary encoder. In some embodiments, the user interface may include a graphical user interface. In some embodiments, the graphical user interface may be configured to display independent patient data for each of the patients. In some embodiments, the user interface may include a touchscreen display. In some embodiments, the ventilator device also may include an enclosure, with the valves, the actuators, the controller, and at least part of the air tube splitter being disposed within the enclosure.

In some embodiments, the ventilator device also may include a ventilator coupled to the inlet of the air tube splitter by a ventilation tubing line and configured to deliver air to the inlet of the air tube splitter. In some embodiments, the controller may be in operable communication with the ventilator and configured to control an air output of the ventilator. In some embodiments, the ventilator may be configured to deliver air to the inlet of the air tube splitter at a flow rate that is at least double a single patient tidal volume requirement. In some embodiments, the ventilator may be configured to deliver air to the inlet of the air tube splitter at a respiratory rate that is at least double a single patient respiratory rate requirement. In some embodiments, the ventilator device also may include a continuous positive airway pressure device coupled to the inlet of the air tube splitter by a ventilation tubing line and configured to deliver air to the inlet of the air tube splitter. In some embodiments, the ventilator device also may include an oxygen tank coupled to the inlet of the air tube splitter by a ventilation tubing line and configured to deliver air to the inlet of the air tube splitter. In some embodiments, the oxygen tank may be configured to deliver air to the inlet of the air tube splitter at a flow rate that is at least double a single patient respiratory rate requirement.

In another aspect, a ventilator system for co-ventilation of multiple patients is provided. In one embodiment, the ventilator system may include a first ventilation device and a second ventilation device each including an air tube splitter having an inlet, a plurality of outlets, and a plurality of branches, with each of the branches extending to a respective one of the outlets. The first ventilator device and the second ventilator device each also may include a plurality of valves coupled to the branches, a plurality of actuators coupled to the valves, and a controller in operable communication with the actuators. Each of the valves may be configured to be adjusted to regulate air flow through a respective one of the branches. Each of the actuators may be configured to adjust a respective one of the valves. The controller may be configured to receive a set of inspiratory pressure settings or tidal volume settings for the patients, and independently control the actuators to adjust the valves based at least in part on the set of inspiratory pressure settings or tidal volume settings. The ventilator system also may include an upstream air tube splitter having an inlet and a plurality of outlets. The inlet of the air tube splitter of the first ventilation device may be fluidically coupled to one of the outlets of the upstream air tube splitter, and the inlet of the air tube splitter of the second ventilation device may be fluidically coupled to another of the outlets of the upstream air tube splitter.

In still another aspect, a method for co-ventilation of multiple patients is provided. In one embodiment, the method may include delivering air from a ventilator to a ventilator device. The ventilator device may include an air tube splitter having an inlet, a plurality of outlets, and a plurality of branches, with each of the branches extending to a respective one of the outlets. The ventilator device also may include a plurality of valves coupled to the branches, a plurality of actuators coupled to the valves, and a controller in operable communication with the actuators. Each of the valves may be configured to be adjusted to regulate air flow through a respective one of the branches. Each of the actuators may be configured to adjust a respective one of the valves. The method also may include receiving, via the controller, a set of inspiratory pressure settings or tidal volume settings for the patients, and independently controlling, via the controller, the actuators to adjust the valves based at least in part on the set of inspiratory pressure settings or tidal volume settings.

In yet another aspect, a method for co-ventilation of a first patient and a second patient is provided. In one embodiment, the method may include delivering air from a ventilator to a ventilator device, delivering air from the ventilator device to the first patient for a first time period while no air is delivered from the ventilator device to the second patient, and delivering air from the ventilator device to the second patient for a second time period while no air is delivered from the ventilator device to the first patient.

In another aspect, a method for co-ventilation of a first patient and a second patient is provided. In one embodiment, the method may include delivering air from a ventilator to a ventilator device, delivering air from the ventilator device to the first patient at a first pressure for a first time period while no air is delivered from the ventilator device to the second patient, and delivering air from the ventilator device to the second patient at a second pressure for a second time period while no air is delivered from the ventilator device to the first patient, with the second pressure being different from the first pressure.

In still another aspect, a method for co-ventilation of a first patient and a second patient is provided. In one embodiment, the method may include delivering air from a ventilator to a ventilator device, delivering a first volume of air from the ventilator device to the first patient over a first time period while no air is delivered from the ventilator device to the second patient, and delivering a second volume of air from the ventilator device to the second patient over a second time period while no air is delivered from the ventilator device to the first patient, with the second volume of air being different from the first volume of air.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1A is a top perspective view of an example ventilator device in accordance with embodiments of the disclosure, showing a plurality of outlets and a plurality of second connectors of a ventilator circuit, a user interface, and an enclosure of the ventilator device.

FIG. 1B is a top perspective view of the ventilator device of FIG. 1A, showing an inlet, the outlets, and the second connectors of the ventilator circuit, the user interface, and the enclosure.

FIG. 1C is a top view of the ventilator device of FIG. 1A, showing the inlet, the outlets, and the second connectors of the ventilator circuit, the user interface, and the enclosure.

FIG. 1D is a side view of the ventilator device of FIG. 1A, showing the inlet, the outlets, and the second connectors of the ventilator circuit, the user interface, and the enclosure.

FIG. 1E is an end view of the ventilator device of FIG. 1A, showing the outlets, the second connectors, and a plurality of valves of the ventilator circuit, the user interface, and the enclosure.

FIG. 1F is a top perspective view of a lower portion of the ventilator device of FIG. 1A, showing the inlet, an air tube splitter, a plurality of first connectors, and the valves of the ventilator circuit disposed within a first portion of the enclosure.

FIG. 1G is a bottom view of an upper portion of the ventilator device of FIG. 1A, showing a plurality of actuators, a controller, a display screen, and a rotary encoder disposed within a second portion of the enclosure.

FIG. 1H is a perspective view of one of the valves of the ventilator circuit of the ventilator device of FIG. 1A, showing the valve configured as a ball valve and positioned in a plurality of different positions.

FIG. 1I is a schematic diagram of a portion of the ventilator device of FIG. 1A, showing the controller in operable communication with the actuators, the user interface and a plurality of sensors of the ventilator device, along with a ventilator.

FIG. 2 is a top view of an example ventilator device in accordance with embodiments of the disclosure, showing three outlets of a ventilator circuit of the ventilator device.

FIG. 3 is a top view of an example ventilator device in accordance with embodiments of the disclosure, showing four outlets of a ventilator circuit of the ventilator device.

FIG. 4 is a schematic diagram of an example air tube splitter for a ventilator device in accordance with embodiments of the disclosure, showing four outlets and four branches of the air tube splitter.

FIG. 5 is a schematic diagram of example air tube splitters for a ventilator device in accordance with embodiments of the disclosure, showing branches of the air tube splitters disposed at different angles relative to one another.

FIG. 6 is a perspective view of an example valve for a ventilator device in accordance with embodiments of the disclosure, showing the valve configured as a pinch valve and positioned in a plurality of different positions.

FIG. 7 is a schematic diagram of an example ventilator device in accordance with embodiments of the disclosure connected to a ventilator, showing a plurality of sensors of the ventilator device.

FIG. 8 is a top view of an example user interface for a ventilator device in accordance with embodiments of the disclosure, showing a display screen and a graphical user interface of the user interface.

FIG. 9 is a top view of an example user interface for a ventilator device in accordance with embodiments of the disclosure, showing a display screen and a graphical user interface of the user interface and electronic devices in wireless communication with the ventilator device.

FIG. 10 is a schematic diagram of an example ventilator device connected to a ventilator and two patients for co-ventilation of the patients in accordance with embodiments of the disclosure.

FIG. 11 is a graph showing an example pressure drop calibration curve for a ventilator device operating in a pressure control ventilation mode in accordance with embodiments of the disclosure.

FIG. 12 is an example of temporal multiplexing provided by a ventilator device operating in a pressure control ventilation mode for co-ventilation of two patients in accordance with embodiments of the disclosure, showing positions of valves of the ventilator device and ventilation waveforms for the patients.

FIG. 13 is an example ventilation schedule for a ventilator device in accordance with embodiments of the disclosure, showing inspiratory times for four patients being co-ventilated using the ventilator device.

FIG. 14 is a schematic diagram of two example ventilator devices connected to a ventilator for co-ventilation of four patients in accordance with embodiments of the disclosure.

FIG. 15 is a schematic diagram of an example ventilator device connected to an oxygen tank for co-ventilation of two patients in accordance with embodiments of the disclosure.

FIG. 16 is a plan view of an example ventilator device connected to an oxygen tank for co-ventilation of two patients in accordance with embodiments of the disclosure, showing a valve and an actuator disposed between the ventilator device and the oxygen tank for controlling a fraction of inspired oxygen for each of the patients.

FIG. 17A is a top perspective view of an example ventilator device in accordance with embodiments of the disclosure, showing a plurality of outlets and a plurality of second connectors of a ventilator circuit, a user interface, and an enclosure of the ventilator device.

FIG. 17B is a top perspective view of a lower portion of the ventilator device of FIG. 17A, showing an inlet, the outlets, an air tube splitter, a plurality of first connectors, a plurality of valves, and the second connectors of the ventilator circuit disposed within a first portion of the enclosure.

FIG. 17C is a bottom perspective view of an upper portion of the ventilator device of FIG. 17A, showing a plurality of actuators, a controller, a display screen, and a rotary encoder disposed within a second portion of the enclosure.

FIG. 17D is a bottom perspective view of the upper portion of the ventilator device of FIG. 17A, showing the ventilator circuit positioned thereon such that the actuators are coupled to the valves of the ventilator circuit.

FIG. 18A is a plan view of an example ventilator in accordance with embodiments of the disclosure, showing a plurality of inspiratory ports, a plurality of expiratory ports, and a user interface of the ventilator.

FIG. 18B is a schematic diagram of an example valve assembly for a ventilator in accordance with embodiments of the disclosure, showing the valve assembly positioned for switching a connection between an inspiratory port of the ventilator and a pair of patient ports for co-ventilation of two patients.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, and/or components have not been described in detail so as not to unnecessarily obscure aspects of the embodiments.

### Overview

Ventilator devices disclosed herein integrate with a medical ventilator to split one airflow into two or more streams with customized pressures, which are then delivered to two or more individual patients, allowing safe co-ventilation of patients of different sizes and different lung compliances, with real-time monitoring and adjustment capability. As described herein, the ventilator device can turn off airflow to a specific patient in a synchronized manner to allow temporal ventilator splitting as a method to allow multiple patients to safely share ventilation. In this manner, the ventilator device may be used to increase the number of patients able to be safely ventilated in situations where ventilator demand exceeds supply, such as during the COVID-19 pandemic, chemical warfare, environmental disasters, mass shootings, or other locoregional disturbances that may cause increased demand. The ventilator device also may help developing countries or areas with limited ventilator resources, where the cost of full ventilators is prohibitive. The ventilator device also may be useful for the military. particularly in remote areas, where appropriate ventilator supply may be limited or logistically difficult because of travel needs.

### Example Ventilator Devices and Methods

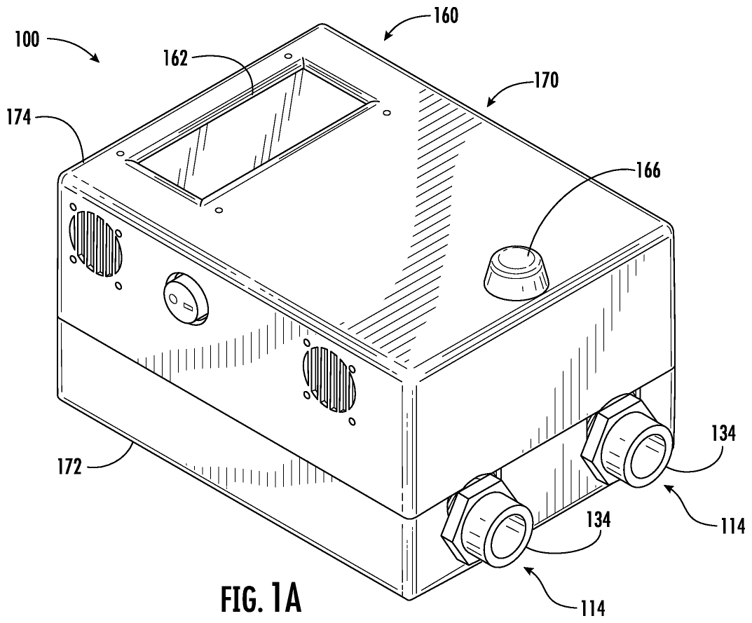

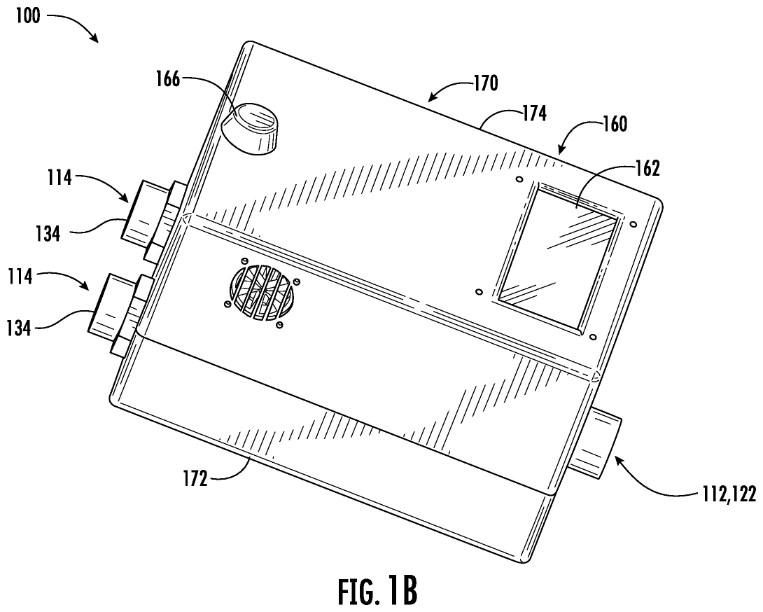

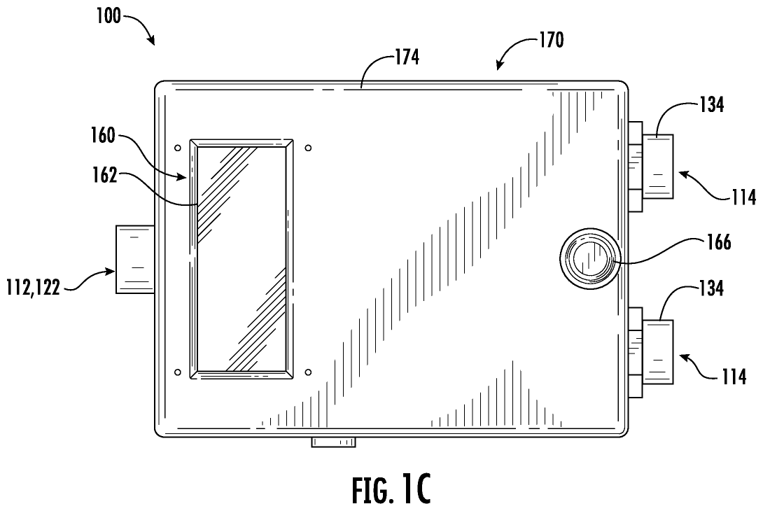

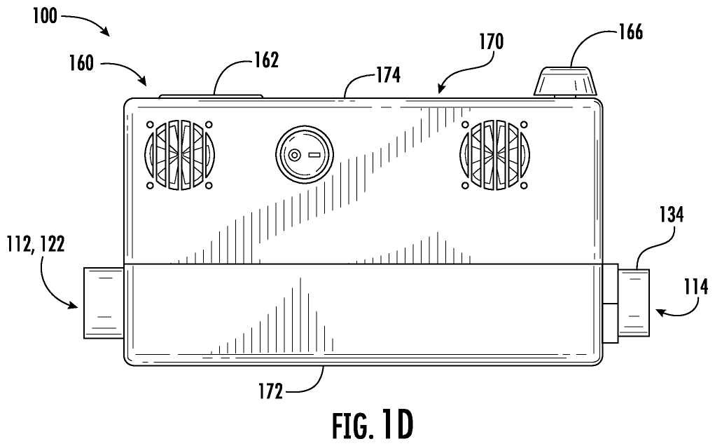

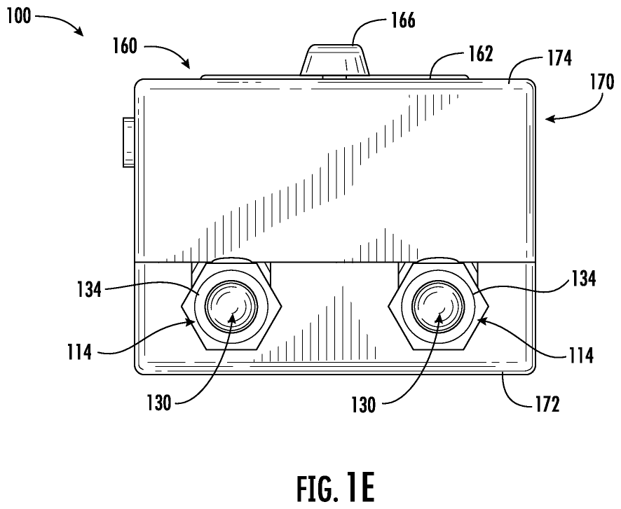

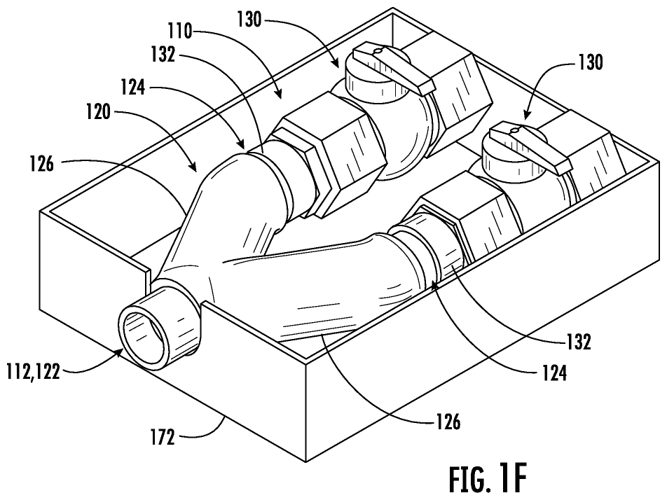

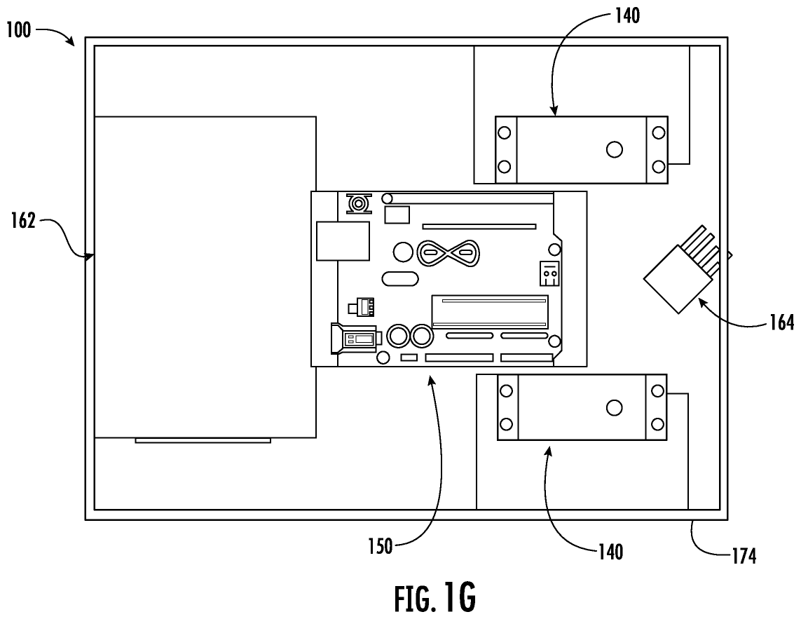

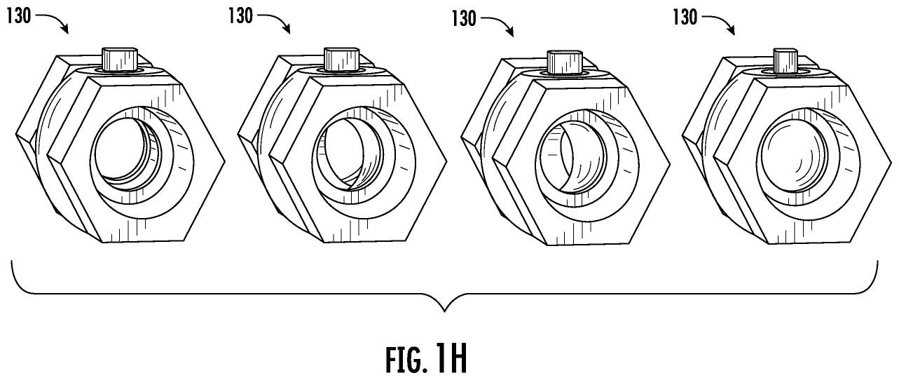

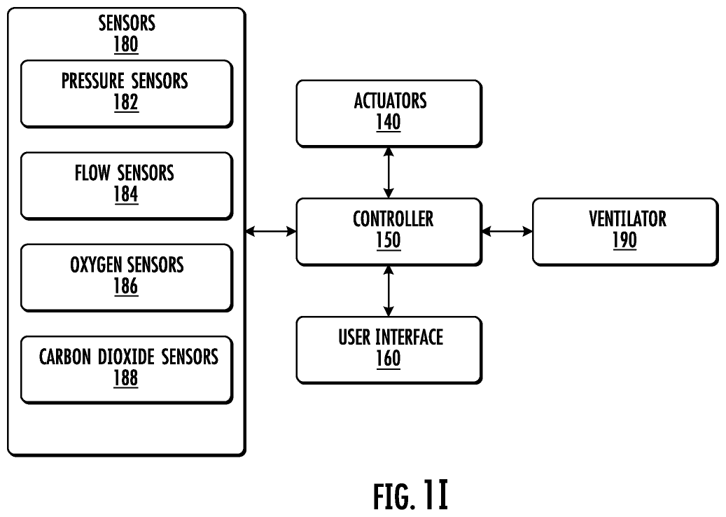

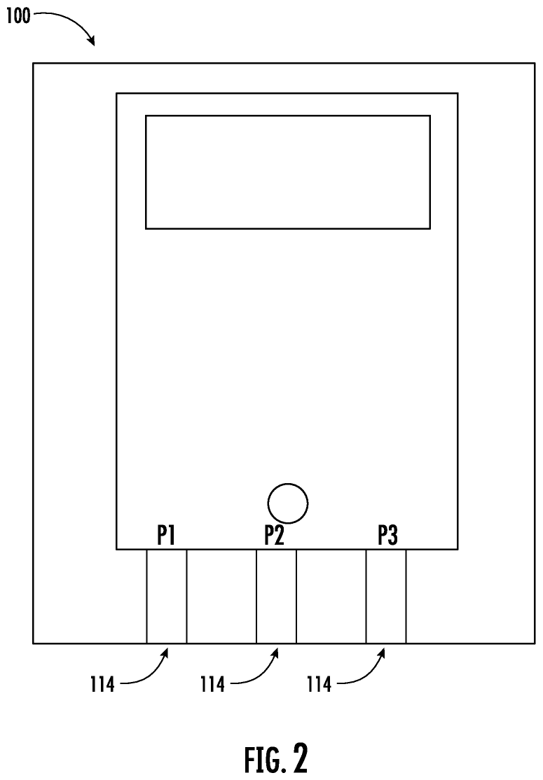

FIGS. 1A-1I depict an example ventilator device 100 and components thereof in accordance with embodiments of the disclosure, which may be used with a ventilator 190 or other source of pressurized air to provide co-ventilation of multiple patients, as described herein. FIGS. 2-9 show example configurations of certain components as may be used as part of the ventilator device 100 in accordance with embodiments of the disclosure.

Referring to FIGS. 1A-1I, the ventilator device 100 generally may include a ventilator circuit 110 having a single inlet 112 and a plurality of outlets 114. During use of the ventilator device 100, the inlet 112 may be connected to the ventilator 190 or other source of pressurized air by a standard ventilator tubing line for receiving air therefrom, and the outlets 114 may be connected to respective patients by additional ventilator tubing lines for delivering air to the patients. According to the illustrated example, the ventilator circuit 110 may include an air tube splitter 120, a plurality of valves 130, a plurality of first connectors 132, and a plurality of second connectors 134. As described further below, the air tube splitter 120 may be configured to divide an incoming flow of air from the ventilator 190 into separate streams, and the valves 130 may be configured to regulate the respective streams of air flowing through the ventilator circuit 110 and ultimately to the respective patients being ventilated. The ventilator device 100 also may include a plurality of actuators 140 and a controller 150 in operable communication with the actuators 140. The actuators 140 may be coupled to the valves 130, with each of the actuators 140 being configured to adjust a respective one of the valves 130. As described further below, the controller 150 may be configured to receive certain settings, such as a set of inspiratory pressure settings or tidal volume settings for the patients, and to independently control the actuators 140 to adjust the valves 130 based at least in part on the settings, such as the set of inspiratory pressure settings or tidal volume settings. The ventilator device 100 also may include a user interface 160 in operable communication with the controller 150. The user interface 160 may be configured to allow a user, such as a clinician, to input and adjust the set of inspiratory pressure settings or tidal volume settings for the patients as well as other settings or operating parameters, as described below. The user interface 160 also may be configured to allow the user to monitor various settings and operating parameters during use of the ventilator device 100. As shown, the ventilator device 100 also may include an enclosure 170) that houses other components of the device 100, such as the valves 130, the actuators 140, the controller 150, and at least part of the air tube splitter 120, therein.

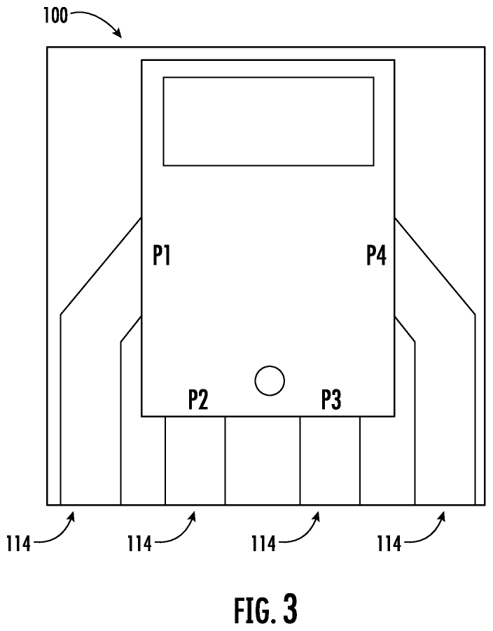

According to the example of FIGS. 1A-1I, the plurality of outlets 114 of the ventilator circuit 110 may consist of two outlets 114. In this manner, during use of the ventilator device 100, the outlets 114 may be connected to two different patients by ventilator tubing lines for delivering air to the patients. In some embodiments, the plurality of outlets 114 may include of three, four, or more outlets 114 for connecting to three, four, or more patients. FIG. 2 shows an example configuration of the ventilator device 100 with the ventilator circuit 110 having three outlets 114, while FIG. 3 shows an example configuration of the ventilator device 100 with the ventilator circuit 110 having four outlets 114. Any number of the outlets 114 suitable for connecting a plurality of patients to a single ventilator may be used in different embodiments, with the number of the outlets 114 generally corresponding to the number of patients intended to be co-ventilated using the ventilator device 100.

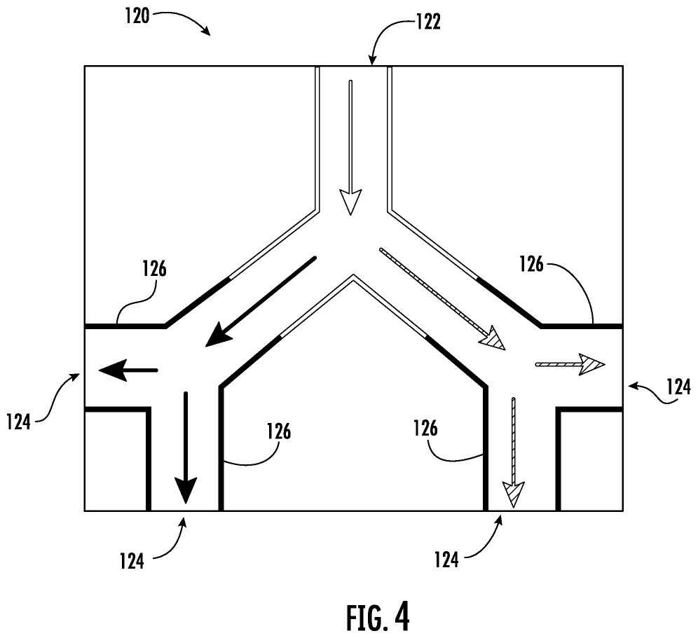

As shown in FIG. 1F, the air tube splitter 120 may include a single inlet 122, a plurality of outlets 124 in fluid communication with the inlet 122, and a plurality of branches 126, with each of the branches 126 extending to a respective one of the outlets 124. In this manner, the air tube splitter 120 may be configured to channel air received from the ventilator 190 and distribute the air among a plurality of ventilation paths to provide co-ventilation of a plurality of patients. In some embodiments, as shown, the inlet 122 of the air tube splitter 120 may define the inlet 112 of the overall ventilator circuit 110. According to the illustrated example, the plurality of outlets 124 may consist of two outlets 124, and the plurality of branches 126 may consist of two branches 126. In some embodiments, the plurality of outlets 124 may include of three, four, or more outlets 124, and the plurality of branches 126 may include three, four, or more branches 126, with the number of the branches 126 corresponding to the number of the outlets 124. For example, FIG. 4 shows an example configuration of the air tube splitter 120 having four outlets 124 and four branches 126. As shown in FIG. 4, the air tube splitter 120 having four outlets 124 may be formed by combining three two-way splitters, which configuration may be used to maintain stable airflow and reduce undesired resistance within the air tube splitter 120. Any number of the outlets 124 and the branches 126 suitable for connecting a plurality of patients to a single ventilator may be used in different embodiments, with the number of the outlets 124 and the branches 126 generally corresponding to the number of patients intended to be co-ventilated using the ventilator device 100.

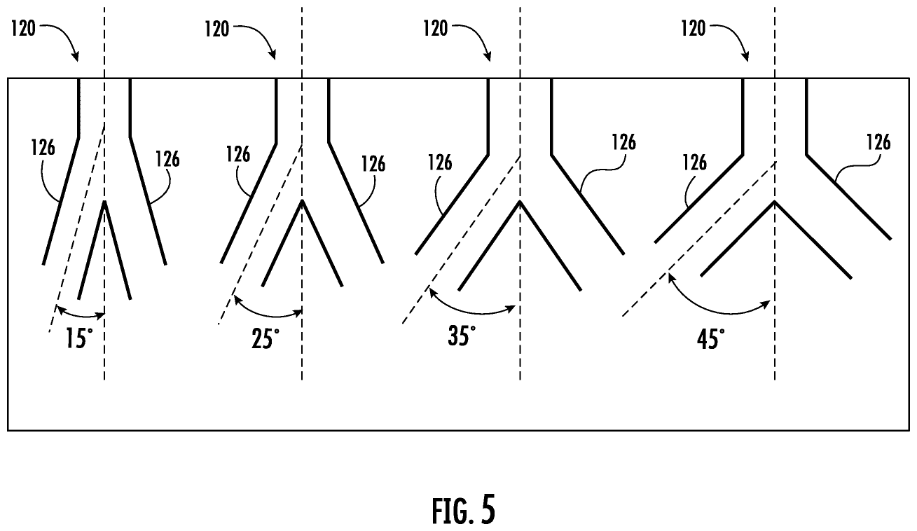

As shown in FIG. 1F, the branches 126 of the air tube splitter 120 may be disposed at an angle with respect to one another, which may be selected to provide desired flow characteristics of air passing through the air tube splitter 120 and the respective branches 126 thereof. In some embodiments, the angle defined between the adjacent branches 126 may be fixed. For example, as shown, the inlets 122, the outlets 124, and the branches 126 of the air tube splitter 120 may be integrally formed with one another and may have a rigid construction, such that the angle between the branches 126 is fixed. In certain embodiments, the angle between the branches 126 may be any angle between 15 degrees and 180 degrees or may be any angle between 15 degrees and 50 degrees. FIG. 5 shows example configurations of the air tube splitter 120 with the branches 126 thereof being disposed at different angles with respect to one another. Various angles may be used in different embodiments to provide desired flow characteristics, to minimize pressure build up, to allow for use with different types of ventilators, and/or to accommodate patients being at different distances from each other. In embodiments in which the air tube splitter 120 includes more than two branches 126, the branches 126 of each adjacent pair of the branches 126 may be disposed at an angle with respect to one another. In some embodiments, the angle defined between the adjacent branches 126 may be adjustable. For example, the air tube splitter 120 may include an adjustable connection disposed between each adjacent pair of the branches 126 and configured to allow adjustment of the angle between the branches 126. In some embodiments, the adjustable connection may include a hinge or other means for allowing adjustment of the angle between the branches 126. In some embodiments, the adjustable connection may be selectively locked to inhibit movement of the branches 126 relative to one another. In some embodiments, the air tube splitter 120 may include markings configured to indicate different angles at which the branches 126 may be adjusted using the adjustable connection. In certain embodiments, the angle between the branches 126 may be adjustable to any angle between 15 degrees and 180 degrees or may be adjustable to any angle between 15 degrees and 50 degrees.

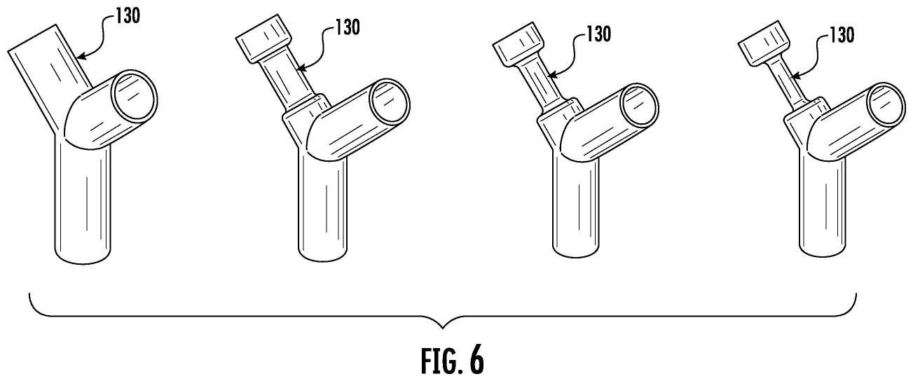

As shown in FIG. 1F, the valves 130 may be configured to regulate air flow through the ventilator circuit 100 to the outlets 114 thereof and ultimately to the plurality of patients being ventilated using the ventilator device 100. In particular, the valves 130 may be used to regulate air flow and provide flow characteristics (e.g., pressure, flow rate, etc.) at each of the outlets 114 in a manner that meets inspiratory pressure requirements and/or tidal volume requirements of the respective patients connected to the ventilator device 100. As shown, each of the valves 130 may be coupled to a respective one of the branches 126 and configured to be adjusted to regulate air flow through the respective branch 126 to the respective outlet 114. According to the illustrated example, the plurality of valves 130 may consist of two valves 130. In some embodiments, the plurality of valves 130 may include three, four, or more valves 130, with the number of the valves 130 corresponding to the number of the branches 126. Any number of the valves 130 suitable for regulating air flow through the ventilator circuit 100 for a plurality of patients may be used in different embodiments, with the number of the valves 130 generally corresponding to the number of patients intended to be co-ventilated using the ventilator device 100. Each of the valves 130 may be configured to be adjusted between an open position (i.e., a fully open position) and a closed position (i.e., a fully closed position). When in the open position, the valve 130 may allow air to freely pass through the respective branch 126 to the respective outlet 114. When in the closed position, the valve 140 may prevent air from passing through the respective branch 126 to the respective outlet 114. In some embodiments, as shown, each of the valves 130 also may be configured to be adjusted to a plurality of partially open positions between the open position and the closed position. In this manner, during use of the ventilator device 100, each of the valves 130 may adjusted to a position suitable for providing desired flow characteristics of air delivered to a respective patient, as described further below. In other words, each of the valves 130 may be adjusted to provide a varying resistance to air flow through a respective portion of the ventilator circuit 110 to a respective outlet 114 thereof. In some embodiments, as shown, each of the valves 130 may be a ball valve. FIG. 1H illustrates one of the valves 130 configured as a ball valve, showing (from left to right) the ball valve in a fully open position, a first partially open position providing a first resistance, a second partially open position providing a second resistance greater than the first resistance, and a fully closed position. In other embodiments, each of the valves 130 may be a pinch valve. FIG. 6 illustrates one of the valves 130 configured as a pinch valve, showing (from left to right) the pinch valve in a fully open position, a first partially open position providing a first resistance, a second partially open position providing a second resistance greater than the first resistance, and a third partially open position providing a third resistance greater than the second resistance. In still other embodiments, each of the valves 130 may be a butterfly valve, a gate valve, or any other suitable type of valve configured to be adjusted between an open position and a closed position to regulate air flow through the ventilator circuit 110.

In some embodiments, the valves 130 may be coupled to the branches 126 of the air tube splitter 120 by the first connectors 132. As shown, the first connectors 132 may be coupled to the branches 126 and the valves 130, with each of the first connectors 132 connecting a respective one of the valves 130 to a respective one of the branches 126. In some embodiments, the second connectors 134 may be coupled to the valves 130 for connecting respective ventilation tubing lines extending between the ventilator device 100 and the patients being ventilated. As shown, the second connectors 134 may be coupled to the valves 130, with each of the second connectors 134 being configured to connect a ventilation tubing line to a respective one of the valves 130. In some embodiments, as shown, the second connectors 134 may define the outlets 114 of the overall ventilator circuit 110. According to the illustrated example, the air tube splitter 120, the valves 130, the first connectors 132, and the second connectors 134 may be separately formed and coupled to one another, as shown, to form the ventilator circuit 110 of the ventilator device 100. In some embodiments, the air tube splitter 120, portions of the valves 130, the first connectors 132, and the second connectors 134 may be integrally formed with one another as a single component. By integrally forming these components, the introduction of undesired resistances or contaminants to the ventilator circuit 110 may be minimized. Other configurations of the ventilator circuit 110 may be used in other embodiments.

The actuators 140 may be coupled to the valves 130 and configured to adjust the positions of the respective valves 130 to regulate air flow through the ventilator circuit 100 in a desired manner for ventilating the patients connected to the ventilator device 100. In particular, each of the actuators 140 may be configured to adjust a respective one of the valves 130 between the open position, closed position, and partially open positions of the valve 130. According to the illustrated example, the plurality of actuators 140 may consist of two actuators 140. In some embodiments, the plurality of actuators 140 may include three, four, or more actuators 140, with the number of the actuators 140 corresponding to the number of the valves 130. Any number of the actuators 140 suitable for adjusting the valves 130 to regulate air flow through the ventilator circuit 100 for a plurality of patients may be used in different embodiments, with the number of the actuators 140 generally corresponding to the number of patients intended to be co-ventilated using the ventilator device 100. As described below, the actuators 140 may be controlled by the controller 150 to adjust the valves 130 in a desired manner. In some embodiments, each of the actuators 140 may be a motor, such as a servo motor, as shown. In other embodiments, each of the actuators 140 may be a solenoid valve or any other type of actuation device suitable for adjusting the valves 130 between the open position, closed position, and partially open positions. In some embodiments, as shown, the actuators 140 may be movably coupled to the valves 130 such that movement of a portion of one of the actuators 140 causes a mating portion of the respective valve 130 to move to adjust the position of the valve 130. For example, rotation of a portion of one of the actuators 140 may cause a mating portion of the respective valve 130 to rotate to adjust the position of the valve 130. Various other types of movable couplings between the actuators 140 and the valves 130 may be used in other embodiments. In some embodiments, as shown, the actuators 140 may be removably coupled to the valves 130, for example, to facilitate disassembly of the ventilator device 100 for maintenance or cleaning purposes.

The controller 150 may be in operable communication with the actuators 140 as well as other electronic components of the ventilator device 100. As shown in FIG. 1I, the controller 150 may be in operable communication with the actuators 140, the user interface 160, and one or more sensors 180 of the ventilator device 100. The controller 150 may be configured to control operation of the actuators 140 to adjust the valves 130 based at least in part on one or more settings and/or one or more operating parameters of the ventilator device 100. In some embodiments, the controller 150 may be configured to receive one or more settings for the patients being treated using the ventilator device 100 and to independently control the actuators 140 to adjust the valves 130 based at least in part on the settings. As described further below, in some embodiments, the controller 150 may be configured to receive a set of inspiratory pressure settings or tidal volume settings for the patients being treated using the ventilator device 100 and to independently control the actuators 140 to adjust the valves 130 based at least in part on the set of inspiratory pressure settings or tidal volume settings. For example, based at least in part on the set of inspiratory pressure settings or tidal volume settings, the controller 150 may send control signals to the respective actuators 140 such that the valves 130 are adjusted to desired positions at desired times in accordance with the inspiratory pressure and/or tidal volume needs of the respective patients. In this manner, the controller 150 may independently control the actuators 140 to adjust the valves 130 to regulate airflow through the ventilator circuit 110 such that each patient receives ventilation having flow characteristics that satisfy the particular patient's inspiratory pressure and/or tidal volume requirements. For example, when air is to be delivered from the ventilator device 100 to a particular patient at a predetermined inspiratory pressure for the patient, the controller 150 may control the respective actuator 140 to adjust the respective valve 130 to the open position or a partially open position, as appropriate, to deliver air to the patient in accordance with the patient's predetermined inspiratory pressure setting. As another example, when a predetermined tidal volume of air is to be delivered from the ventilator device 100 to a particular patient, the controller 150 may control the respective actuator 140 to adjust the respective valve 130 to the open position or a partially open position for a period of time and then to the closed position, as appropriate, to deliver a volume of air to the patient in accordance with the patient's predetermined tidal volume setting. As described below, the set of inspiratory pressure settings or tidal volume settings may be input by a user, such as a clinician, via the user interface 160 and provided to the controller 150.

In some embodiments, the controller 150 may be configured to receive a predetermined inspiratory to expiratory ratio for the patients being treated using the ventilator device 100 and to independently control the actuators 140 to adjust the valves 130 based at least in part on the predetermined inspiratory to expiratory ratio for the patients. In some embodiments, the controller 150 may be configured to receive a predetermined inspiratory to expiratory ratio of the ventilator 190 being used with the ventilator device 100 and to independently control the actuators 140 to adjust the valves 130 based at least in part on the predetermined inspiratory to expiratory ratio of the ventilator. As described below; the predetermined inspiratory to expiratory ratio for the patients and the predetermined inspiratory to expiratory ratio of the ventilator may be input by a user, such as a clinician, via the user interface 160 and provided to the controller 150.

In some embodiments, the controller 150 may employ temporal multiplexing in independently controlling the actuators 140 to adjust the valves 130 for providing ventilation to a plurality of patients. As described further below, the valves 130 may be alternately adjusted such that only one of the valves 130 is in the open position or a partially open position while a remainder of the valves 130 are in the closed position, and thus air is delivered from the ventilator device 100 to only one of the patients at a time. For example, during operation of the illustrated ventilator device 100 having two valves 130, the first valve 130 may be adjusted to the open position or a partially open position to deliver air to a first patient while the second valve 130 is in the closed position for a first time period, and then the second valve 130 may be adjusted to the open position or a partially open position to deliver air to a second patient while the first valve 130 is in the closed position for a subsequent second time period. Further, for other time periods during which the ventilator 190 is not delivering air to the ventilator device 100, both of the valves 130 may be maintained in the closed position, such that no air is delivered from the ventilator device 100 to the patients during those time periods. According to the temporal multiplexing approach, the controller 150 may determine the timing and duration of the time periods during which one of the valves 130 is in the open position or a partially open position and the time periods during which both of the valves 130 are maintained in the closed position based at least in part on the predetermined inspiratory to expiratory ratio for the patients. In various embodiments, the predetermined inspiratory to expiratory ratio for the patients may be 1:1, 1:1.5, 1:2, 1:3, 1:4, 1:5, or any other ratio suitable for providing ventilation to the plurality of patients being ventilated using the ventilator device 100. It will be appreciated that the predetermined inspiratory to expiratory ratio for the patients selected by a clinician may be influenced by the range of inspiratory to expiratory ratios at which the ventilator 190 being used with the ventilator device 100 is capable of operating. In some embodiments, the controller 150 may be an Arduino microcontroller. In other embodiments, the controller 150 may be a Raspberry Pi controller, which may use Python programming language. In still other embodiments, the controller 150 may be any processor capable of independently controlling the actuators 140 to adjust the valves 130 for providing ventilation to a plurality of patients. The controller 150 may be programmed to receive one or more settings and/or one or more operating parameters, as described herein, and to control the actuators 140 to adjust the valves based at least in part on the one or more settings and/or one or more operating parameters.

As shown in FIG. 1I, in some embodiments, the sensors 180 of the ventilator device 100 may include a plurality of pressure sensors 182. The pressure sensors 182 may be coupled to the branches 126 of the air tube splitter 120 downstream from the valves 130 and in operable communication with the controller 150, with each of the pressure sensors 182 being configured to detect a pressure of air flow downstream from a respective one of the valves 130. The controller 150 may be configured to receive pressure signals indicative of pressures detected by the pressure sensors 182 and to independently control the actuators 140 to adjust the valves 130 based at least in part on the pressure signals. In this manner, the controller 150 may be configured to determine a pressure of air being delivered to a particular patient and to control the respective actuator 140 to adjust the respective valve 130, as needed, in accordance with a predetermined inspiratory pressure setting for the patient. In some embodiments, the controller 150 may be configured to activate an alarm, such as an audible alarm, or to cause the user interface 160 to display a warning message if the pressure detected by one of the pressure sensors 182 is outside of a predetermined range for the respective patient.

In some embodiments, the sensors 180 of the ventilator device 100 may include a plurality of flow sensors 184, as shown in FIG. 1I. The flow sensors 184 may be coupled to the branches 126 of the air tube splitter 120 downstream from the valves 130 and in operable communication with the controller 150, with each of the flow sensors 184 being configured to detect a flow rate of air flow downstream from a respective one of the valves 130. The controller 150 may be configured to receive flow rate signals indicative of flow rates detected by the flow sensors 184 and to independently control the actuators 140 to adjust the valves 130 based at least in part on the flow rate signals. In this manner, the controller 150 may be configured to determine a flow rate of air being delivered to a particular patient and to control the respective actuator 140 to adjust the respective valve 130, as needed, in accordance with a predetermined tidal volume setting or flow rate setting for the patient. In some embodiments, the controller 150 may be configured to activate an alarm, such as an audible alarm, or to cause the user interface 160 to display a warning message if the flow rate detected by one of the flow sensors 184 is outside of a predetermined range for the respective patient.

In some embodiments, the sensors 180 of the ventilator device 100 may include a plurality of oxygen sensors 186, as shown in FIG. 1I. The oxygen sensors 186 may be coupled to the branches 126 of the air tube splitter 120 downstream from the valves 130 and in operable communication with the controller 150, with each of the oxygen sensors 186 being configured to detect an oxygen concentration of air flow downstream from a respective one of the valves 130. The controller 150 may be configured to receive oxygen concentration signals indicative of oxygen concentrations detected by the oxygen sensors 186 and to independently control the actuators 140 to adjust the valves 130 based at least in part on the oxygen concentration signals. In this manner, the controller 150 may be configured to determine an oxygen concentration of air being delivered to a particular patient and to control the respective actuator 140 to adjust the respective valve 130, as needed, in accordance with a predetermined oxygen concentration setting for the patient. In some embodiments, the controller 150 may be configured to activate an alarm, such as an audible alarm, or to cause the user interface 160 to display a warning message if the oxygen concentration detected by one of the oxygen sensors 186 is outside of a predetermined range for the respective patient.

In some embodiments, the sensors 180 of the ventilator device 100 may include a plurality of carbon dioxide sensors 188, as shown in FIG. 1I. The carbon dioxide sensors 188 may be coupled to the branches 126 of the air tube splitter 120 downstream from the valves 130 and in operable communication with the controller 150, with each of the carbon dioxide sensors 188 being configured to detect a carbon dioxide concentration of air flow downstream from a respective one of the valves 130. The controller 150 may be configured to receive carbon dioxide concentration signals indicative of carbon dioxide concentrations detected by the carbon dioxide sensors 188 and to independently control the actuators 140 to adjust the valves 130 based at least in part on the carbon dioxide concentration signals. In this manner, the controller 150 may be configured to determine a carbon dioxide concentration of air being delivered to a particular patient and to control the respective actuator 140 to adjust the respective valve 130, as needed, in accordance with a predetermined carbon dioxide concentration setting for the patient. In some embodiments, the controller 150 may be configured to activate an alarm, such as an audible alarm, or to cause the user interface 160 to display a warning message if the carbon dioxide concentration detected by one of the carbon dioxide sensors 188 is outside of a predetermined range for the respective patient.

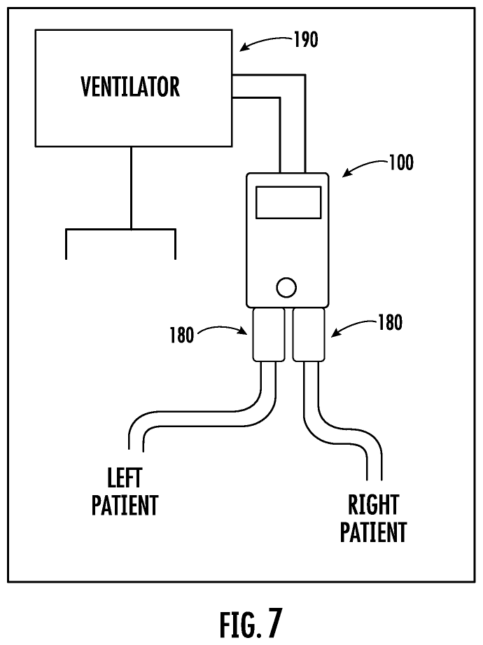

FIG. 7 shows an example configuration of the sensors 180 of the ventilator device 100, which may include the pressure sensors 182, the flow sensors 184, the oxygen sensors 186, and/or the carbon dioxide sensors 188 described above as well as additional sensors configured to detect additional operating parameters. In some embodiments, as shown, the sensors 180 may be disposed downstream from the valves 130, with each set of the sensors 180 being configured to detect relevant operating parameters of air flow downstream from a respective one of the valves 130. In this manner, the operating parameters of air flowing to each of the patients may be determined by the sensors 180 and monitored by the controller 150 to identify a need to adjust the valves 130 to meet ventilation requirements of the respective patients. Various other configurations of the sensors 180 may be used in other embodiments.

As shown in FIG. 1I, in some embodiments, the controller 150 also may be in operable communication with the ventilator 190 or other source of pressurized air being used with the ventilator device 100. In this manner, the controller 150 may be configured to communicate directly with the ventilator 190. For example, the controller 150 may be configured to receive one or more settings and/or one or more operating parameters of the ventilator 190 from the ventilator 190 and/or the controller 150 may be configured to communicate one or more settings and/or one or more operating parameters of the ventilator device 100 to the ventilator 190. In some embodiments, the controller 150 may be configured to modify one or more settings and/or one or more operating parameters of the ventilator device 100 based at least in part on one or more settings and/or one or more operating parameters of the ventilator 190. In some embodiments, the controller 150 may be configured to cause the ventilator 190 to modify one or more settings and/or one or more operating parameters of the ventilator 190 based at least in part on one or more settings and/or one or more operating parameters of the ventilator device 100. In some embodiments, the ventilator 190 may be configured to deliver air to the ventilator device 100 at a respiratory rate that is at least double a single patient respiratory rate requirement, thereby providing sufficient air for ventilation of at least two patients. In some embodiments, the ventilator 190 may be a Philips Respironics V60 ventilator, although other types of ventilators suitable for providing enough air for ventilating a plurality of patients may be used in other embodiments.

As shown in FIG. 1I, the user interface 160 may be in operable communication with the controller 150. The user interface 160 may be configured to allow a user, such as a clinician, to input and adjust one or more settings and/or one or more operating parameters of the ventilator device 100 and/or the ventilator 190, and to communicate such settings and/or operating parameters to the controller 150. For example, the user interface 160 may be configured to allow a user to input and adjust one or more settings for the patients being ventilated using the ventilator device 100, such as a set of inspiratory pressure settings or tidal volume settings for the patients, an inspiratory to expiratory ratio for the patients, and/or other settings for the patients. The user interface 160 also may allow a user to input and adjust one or more settings of the ventilator 190 being used with the ventilator device 100, such as an inspiratory pressure setting of the ventilator 190, an inspiratory to expiratory ratio of the ventilator 190, and/or other settings of the ventilator 190. In some embodiments, the user interface 160 may be configured to display the one or more settings and/or one or more operating parameters of the ventilator device 100 and/or the ventilator 190, such that a user may monitor the settings and/or operating parameters.

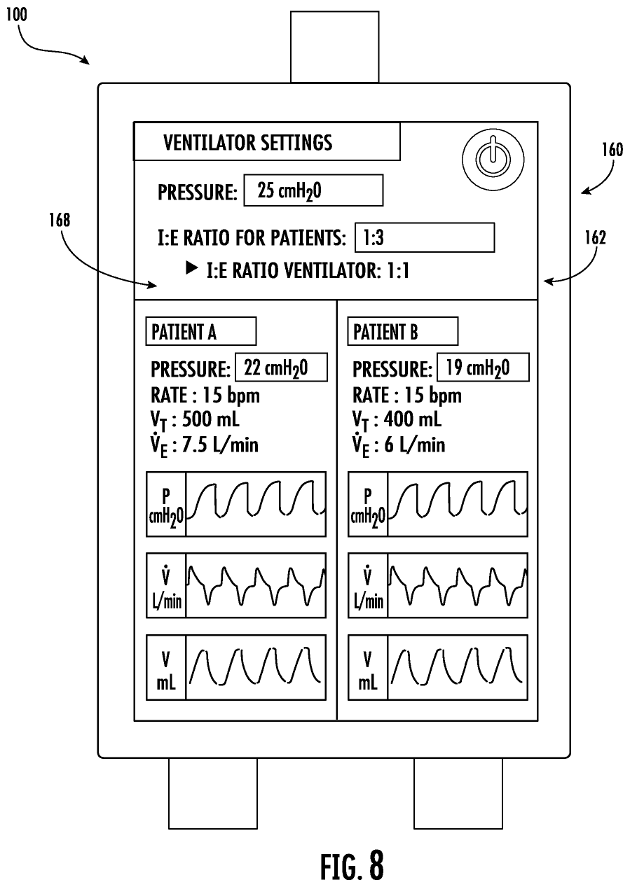

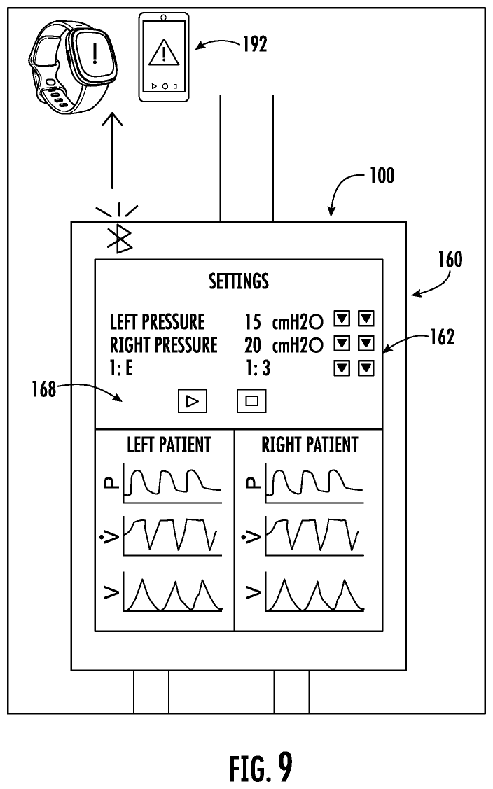

As shown in FIGS. 1A-1G, the user interface 160 may include a display screen 162, a rotary encoder 164, and a knob 166 coupled to the rotary encoder 164. The display screen 162 may be configured to display the one or more settings and/or one or more operating parameters of the ventilator device 100 and/or the ventilator 190 discussed above. In some embodiments, the display screen 162 may be a liquid-crystal display (LCD) screen, although other suitable types of display screens may be used in other embodiments. The knob 166 may be configured to be rotated, depressed, and/or otherwise manipulated by a user to allow the user to input and adjust the one or more settings and/or one or more operating parameters via the rotary encoder 164. Various other configurations of the user interface 160 may be used in different embodiments. FIG. 8 shows an example configuration of the user interface 160 having the display screen 162 as a touchscreen display. In this manner, a user may interact directly with the display screen 162 to adjust settings and/or operating parameters, obviating the need for a knob or other input device. The display screen 162 may provide a graphical user interface (GUI) 168 that displays various settings and operating parameters of the ventilator device 100 for the user. As shown, the GUI 168 may display independent patient data for each of the patients being treated using the ventilator device 100. FIG. 9 shows another example configuration of the user interface 160 having the display screen 162 as a touchscreen display and providing the GUI 168 for the user. As shown, the GUI 168 may display independent patient data for each of the patients being treated using the ventilator device 100. As shown, the user interface 160 may be in wireless communication with one or more additional electronic devices 192, such as a smartphone or a smartwatch, for example, via a wireless receiver implementing Bluetooth or another form of wireless communication. In this manner, the GUI 168 also may be displayed on the additional electronic device(s) 192 to allow the user, such as a clinician, to view, input, or adjust settings or operating parameters of the ventilator device 100. In some embodiments, the user interface 160 may communicate alerts or warning messages to the additional electronic device(s) 192, when settings or operating parameters of the ventilator device 100 require the user's attention, as discussed above. In some embodiments, the user interface 160, or more specifically the GUI 168, may include a locking feature configured to restrict operation of the user interface 160 to only authorized users, such as authorized clinicians. For example, the user interface 160 may require a passcode, fingerprint identification, or facial identification in order to unlock the user interface 160 and allow a user to input or adjust settings or operating parameters.

The electronic components of the ventilator device 100, including the actuators 140, the controller 150, the user interface 160, and the sensors 180, may be powered by an external power source. For example, the ventilator device 100 may be connected to mains power for providing power to the electronic components. In some embodiments, the ventilator device 100 may include an internal power source, such as one or more batteries or other power storage devices, as a backup to the external power source. In this manner, the internal power source may allow for temporary use of the ventilator device 100 while external power is not available, for example, during intermittent power outages. Because the ventilator device 100 is intended for use in emergency situations, the internal power source may be particularly beneficial for patient safety, enabling continuous operation of the ventilator device 100.

As shown in FIGS. 1A-1G, the enclosure 170 may be configured to house other components of the ventilator device 100 therein. The enclosure 170 may protect the enclosed components and allow the ventilator device 100 to be safely and conveniently transported and installed in various applications. According to the illustrated example, the enclosure 170) may house the valves 130, the actuators 140, the controller 150, and at least part of the air tube splitter 120 therein. As shown, the inlet 122 of the air tube splitter 120 may protrude from one end of the enclosure 170 for connection to the ventilator 190 via a ventilation tubing line, and the second connectors 134 may protrude from the other end of the enclosure 170 for connection to the patients via additional ventilation tubing lines. In some embodiments, as shown, the enclosure 170 may include a first portion 172 and a second portion 174 that are removably coupled to one another. The first portion 172 and the second portion 174 may be coupled to one another during use of the ventilator device 100 and may be decoupled to allow the enclosure 170 to be opened, for example, to facilitate maintenance or cleaning of the internal components. Various types of mechanical fasteners may be used to removably couple the first portion 172 and the second portion 174 to one another. In some embodiments, as shown, the first portion 172 may house at least part of the air tube splitter 120, the valves 130, and the first connectors 132 therein, and the second portion 174 may house at least part of the actuators 140, the controller 150, the display screen 162, and the rotary encoder 164 therein. Various other configurations of the enclosure 170 may be used in other embodiments.

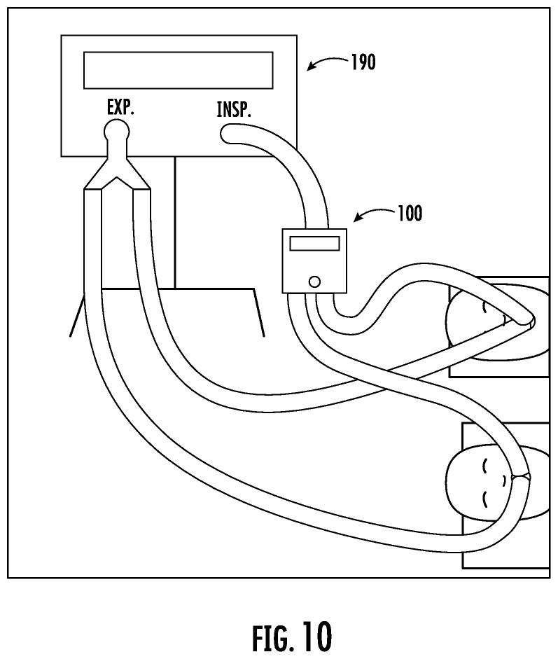

FIG. 10 illustrates an example use of the ventilator device 100 and the ventilator 190 for co-ventilation of two patients. As shown, the ventilator device 100 may be connected to the ventilator 190 by a standard ventilator tubing line extending from the inspiratory port of the ventilator 190 to the inlet 112 of the ventilator circuit 110 of the ventilator device 100. In this manner, a single flow of air may be delivered from the ventilator 190 to the ventilator device 100. The ventilator device 100 may be connected to the patients by additional ventilator tubing lines extending from the respective outlets 114 of the ventilator circuit 110 to the respective patients. In this manner, respective flows of air may be delivered from the ventilator device 100 to the respective patients. In some embodiments, as shown, additional ventilator tubing lines may extend from the respective patients to the expiratory port of the ventilator 190. The clinician may determine appropriate settings of the ventilator 190, including an inspiratory pressure setting and an inspiratory to expiratory ratio of the ventilator 190, and input the settings to both the ventilator 190 and the ventilator device 100. The clinician also may determine appropriate settings for the ventilator device 100, including a set of inspiratory pressure settings or tidal volume settings for the patients and an inspiratory to expiratory ratio for the patients, and input the settings to the ventilator device 100. Before connecting the patients to the ventilator device 100 via the ventilator tubing lines, the ventilator device 100 and the ventilator 190 may be started to allow for synchronization. An inspiratory trigger of the ventilator 190 may provide the ability for the ventilator 190 to sense when each of the valves 130 for the respective patients is opened to room air. For embodiments in which the ventilator device 100 is configured as an accessory to the ventilator 190, as shown, the inspiratory trigger of the ventilator 190 may be required to accomplish synchronization. Once synchronization is obtained, the ventilator tubing lines extending from the ventilator device 100 may be connected to the patients. In some embodiments, unidirectional valves (i.e., one-way valves) may be provided between the ventilator device 100 and each of the patients to maintain synchronization. Specifically, the unidirectional valves may prevent a spontaneous breath from either patient from disturbing the synchronization and the ventilation of the other patient. Furthermore, the unidirectional valves may allow each of the patients to breathe spontaneously from room air while their branch of the ventilator circuit 110 is closed to the ventilator 190.

The ventilator device 100 may be configured to operate in different modes for controlling ventilation of a plurality of patients. In some embodiments, the ventilator device 100 may be configured to operate in a pressure control ventilation mode, according to which the controller 150 controls the actuators 140 to adjust the valves 130 to regulate the pressure of air delivered to the respective patients based on settings input by the clinician for the different patients. Specifically, the clinician may use the user interface 160 to input a set of inspiratory pressure settings for the patients, including a particular inspiratory pressure setting for each of the respective patients, as well as an inspiratory pressure setting of the ventilator 190 (i.e., the pressure of air delivered from the ventilator 190 to the ventilator device 100). The controller 150 may receive the set of inspiratory pressure settings for the patients and the inspiratory pressure setting of the ventilator 190 from the user interface 160 and may independently control the actuators 140 to adjust the valves 130 based at least in part on the set of inspiratory pressure settings for the patients and the inspiratory pressure setting of the ventilator 190. In particular, the controller 150 may control the actuators 140 to adjust the valves 130 such that the respective resistances provided by the valves 130 correspond to the respective inspiratory pressure settings for the patients. The resistance provided by each of the valves 130 may induce a pressure drop from the pressure of air being delivered from the ventilator 190 to the ventilator device 100 to a lower pressure for the respective patient, thereby allowing for safe delivery of appropriate pressures and volumes for each of the patients. In this manner, each of the patients may receive customized breaths, despite the patients having different ventilation requirements. During operation of the ventilator device 100, the resistance being provided by a particular one of the valves 130 may be changed by the clinician adjusting the inspiratory pressure setting for the respective patient via the user interface 160. In this manner, the ventilator device 100 may provide dynamic control of the pressures of air being delivered to the patients, allowing the ventilator device 100 to adapt to changing ventilation needs of each patient as that patient's condition worsens or improves, without impacting the ventilator device's ability to provide adequate ventilation to the remaining patients. As discussed above, the clinician also may use the user interface 160 to input an inspiratory to expiratory ratio for the patients and an inspiratory to expiratory ratio of the ventilator 190, which the controller 150 may use to split airflow temporally to the different patients being ventilated using the ventilator device 100.

In some embodiments, the controller 150 may control the actuators 140 to adjust the valves 130 based at least in part on a predetermined pressure drop calibration curve for the valves 130. FIG. 1I shows an example pressure drop calibration curve for one of the valves 130 implemented as a ball valve. According to the example, the pressure drop associated with various positions (i.e., closed position, open position, and various partially open positions) of the valve 130 was experimentally determined and programmed into the controller 150 of the ventilator device 100. This was accomplished by correlating the tidal volumes delivered at a fixed pressure and different resistances (i.e., different resistances provided by the valve being positioned at different ball valve angles) to tidal volumes delivered at various pressures with no resistance (i.e., with the valve in the fully open position). The resulting curve may be implemented into the code of the controller 150, allowing the controller 150 to determine a position to which the valve 130 should be adjusted during the inspiratory phase for the respective patient. As discussed above, using the user interface 160 of the ventilator device 100, the inspiratory pressure setting for each patient may be safely adjusted at any point during ventilation, with the controller 150 using the pressure drop calibration curve to determine the appropriate position to which the respective valve 130 should be opened based on the adjusted inspiratory pressure setting for the patient. In this manner, the ventilator device 100 allows for independent dynamic pressure control of air being delivered to each of the patients.

As discussed above, in some embodiments, the ventilator device 100 may include pressure sensors 182 in operable communication with the controller 150, with each of the pressure sensors 182 being configured to detect a pressure of air flow downstream of a respective one of the valves 130. The controller 150 may be configured to receive pressure signals indicative of pressures detected by the pressure sensors 182 and to independently control the actuators 140 to adjust the valves 130 based at least in part on the pressure signals. In this manner, one of the pressure sensors 182 may be used to detect the pressure of air being delivered to one of the patients during that patient's inspiratory phase, and the controller 150 may control the respective actuator 140 to adjust the respective valve 130 based on a difference between the pressure detected by the pressure sensor 182 and the inspiratory pressure setting for the patient. For example, if the detected pressure is greater than the patient's inspiratory pressure setting, the controller 150 may control the actuator 140 to adjust the valve 130 to a partially open position closer to the closed position. Conversely, if the detected pressure is less than the patient's inspiratory pressure setting, the controller 150 may control the actuator 140) to adjust the valve 130 to a partially open position closer to the open position. In this manner, the controller 150 and the respective pressure sensor 182 may implement a near real-time feedback loop for controlling the pressure of air being delivered to the respective patient.

In some embodiments, the controller 150 may use a pre-determined pressure drop calibration curve, as discussed above, along with the pressure signals received from the pressure sensors 182 in controlling the actuators 140 to adjust the valves 130. Specifically, the controller 150 may implement code for using the detected pressure data to override the pre-determined pressure drop calibration curve to ensure accuracy of the ventilation provided to the respective patients. In this manner, the controller 150 may implement a near real-time feedback loop for controlling the pressure of air being delivered to the respective patients. As discussed above, the pressure sensors 182 may be coupled to the branches 126 of the air tube splitter 120 downstream from the valves 130 and in operable communication with the controller 150, with each of the pressure sensors 182 being configured to detect a pressure of air flow downstream from a respective one of the valves 130. The controller 150 may be configured to receive pressure signals indicative of pressures detected by the pressure sensors 182 and to independently control the actuators 140 to adjust the valves 130 based at least in part on the pressure signals. In this manner, the controller 150 may be configured to determine a pressure of air being delivered to a particular patient and to control the respective actuator 140 to adjust the respective valve 130, as needed, in accordance with a predetermined inspiratory pressure setting for the patient, as input by the clinician. In an example scenario, the inspiratory pressure setting for a patient may be set at 21 cmH₂O, while the pressure sensor 182 corresponding to the patient may detect that the pressure of air delivered to the patient is only 19.2 cmH₂O (e.g., due to a leak in the circuit or variation in the consistency of breaths delivered by the ventilator 190). In this scenario, based on the detected pressure, the controller 150 may control the respective actuator 140 to adjust the respective valve 130 to a position that more accurately corresponds to the inspiratory pressure setting for a patient (i.e., adjusting the valve 130 to a position closer to the fully open position). In some embodiments, the controller 150 may use an adjustment algorithm that receives the current position of the valve 130 and the detected pressure as inputs and that provides an output of a new position to which the valve 130 is to be moved. In some embodiments, the controller 150 may rely solely on the pressure signals received from the pressure sensors 182 in controlling the actuators 140 to adjust the valves 130, eliminating the need for a pressure drop calibration curve. In some embodiments, the controller 150 may rely solely on a pressure drop calibration curve in controlling the actuators 140 to adjust the valves 130, for example, when the ventilator device does not include the pressure sensors 182. Still other approaches for operating the ventilator device 100 in the pressure control ventilation mode may be used in other embodiments.

In some embodiments, the controller 150 may be configured to use data from any of the sensors 180 or any combination of the sensors 180 in implementing a near real-time feedback loop for controlling one or more characteristics of air being delivered to the respective patients. For example, the controller 150 may use pressure data, flow rate data, tidal volume data, or lung compliance data, or any combination of such data, in implementing a near real-time feedback loop for controlling one or more characteristics of air being delivered to the respective patients. In some embodiments, the controller 150 may use data for non-airflow related parameters, such as blood oxygen levels, end tidal CO2 concentration, blood pressure, heart rate, or other physiological measurements in implementing a near real-time feedback loop for controlling one or more characteristics of air being delivered to the respective patients. In implementing any of these types of feedback loops, the controller 150 may use one or more algorithms that receive the corresponding data as an input and that provide an output corresponding to a particular position of the respective valve 130 for a particular patient such that the patient receives air having the desired characteristics.

In some embodiments, the controller 150 may be configured to perform an internal calibration using data from one or more of the sensors 180. For example, the internal calibration may be performed with test lungs in a simulated scenario of co-ventilation. The controller 150 may receive the settings of the ventilator 190, input by a user via the user interface 160, and the corresponding data signals from the sensors 180 obtained during ventilation of the test lungs with the valves 130 in various positions. The controller 150 may use the detected data to generate a new calibration curve that the controller 150 then uses for controlling the actuators 140 to adjust the valves 130 during subsequent operation of the ventilator device 100 for co-ventilation of multiple patients. Such internal calibration may be particularly useful in providing compatibility of the ventilator device 100 with many different types of ventilators 190.

As discussed above, in some embodiments, the controller 150 may employ temporal multiplexing in independently controlling the actuators 140 to adjust the valves 130 for providing ventilation to a plurality of patients. Specifically, the valves 130 may be alternately adjusted such that only one of the valves 130 is in the open position or a partially open position while a remainder of the valves 130 are in the closed position, and thus air is delivered from the ventilator device 100 to only one of the patients at a time. The temporal multiplexing approach may be implemented based on the inspiratory to expiratory ratio of the ventilator 190 and the inspiratory to expiratory ratio for the patients being ventilated using the ventilator device 100. The respiratory rates of patients consist of an inspiratory time (I-time) and an expiratory time (E-time). During the inspiratory time for a particular patient, which may be one second in duration, the ventilator device 100 may deliver air to the patient at a set pressure or volume. During the expiratory time for a particular patient, which may be of a variable duration depending on the number of patients being co-ventilated using the ventilator device 100, the patient may exhale passively while no air is being delivered from the ventilator device 100 to the patient. The time multiplexing approach takes advantage of this resting time to deliver another personalized breath to a second patient. To accomplish this, the ventilation rate of the ventilator 190 may be doubled. In this manner, the ventilator device 100 may facilitate the distribution and customization of each breath to each patient in an alternating pattern.

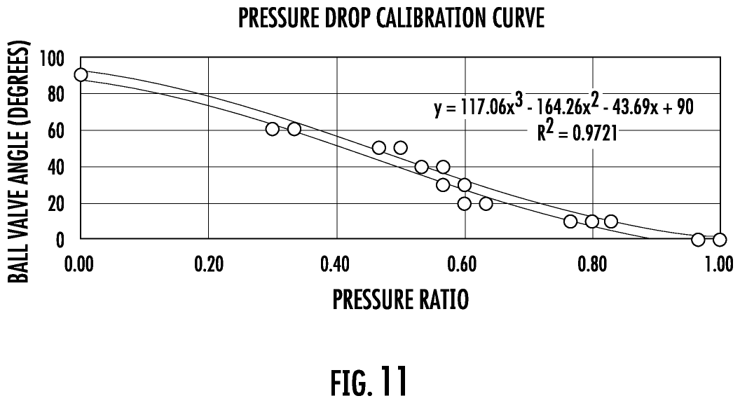

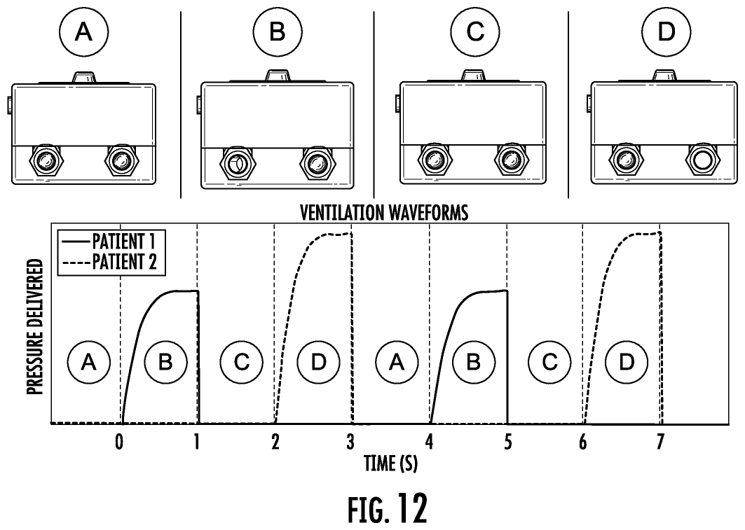

FIG. 12 shows an example of the temporal multiplexing approach as may be employed by the ventilator device 100 operating in the pressure control ventilation mode for co-ventilation of two patients. According to the illustrated example, the ventilator 190 may be programmed to deliver breaths with an inspiratory to expiratory ratio of 1:1, and the ventilator device 100 may mediate the delivery of customized breaths to each of the two patients, providing each patient with an inspiratory to expiratory ratio of 1:3. As discussed above, this approach allows for the independent manipulation of the pressure waveforms being delivered to each patient. As shown, the ventilator device 100 may proceed through a set of four stages. identified as stage A, stage B, stage C, and stage D, in a repeating manner, with each stage having a duration of one second. FIG. 12 shows how the valves 130 of the ventilator device 100 are positioned in the different stages as well as the corresponding ventilation waveforms for the two patients. During stage A, both of the valves 130 may be in the closed position, as shown, such that no air is delivered from the ventilator device 100 to either of the patients. During stage B, the first valve 130 (i.e., the left valve 130 in FIG. 11) may be in a partially open position while the second valve 130 (i.e., the right valve 130 in FIG. 11) is in the closed position, as shown, such that air is delivered from the ventilator device 100 to the first patient, while no air is delivered from the ventilator device 100 to the second patient. During stage C, both of the valves 130 may be in the closed position, as shown, such that no air is delivered from the ventilator device 100 to either of the patients. During stage D, the second valve 130 may be in the open position while the first valve 130 is in the closed position, as shown, such that air is delivered from the ventilator device 100 to the second patient, while no air is delivered from the ventilator device 100 to the first patient. In this manner, stages A and C correspond to the expiratory phase of the ventilator 190, during which no air is being delivered from the ventilator 190 to the ventilator device 100, while stages B and D correspond to the inspiratory phase of the ventilator 190, during which air is being delivered from the ventilator 190 to the ventilator device 100. As shown, stage B corresponds to the inspiratory phase for the first patient, while stages C, D, and A correspond to the expiratory phase for the first patient. Meanwhile, stage D corresponds to the inspiratory phase for the second patient, while stages A, B, and C correspond to the expiratory phase for the second patient. As discussed above, when operating in the pressure control ventilation mode, the ventilator device 100 may deliver air at different pressures to the respective patients. According to the illustrated example, the pressure of the air delivered to the first patient during stage B may be less than the pressure of the air delivered to the second patient during stage D. As discussed above, the pressure of air delivered from the ventilator device 100 to a particular patient may be determined by the position to which the respective valve 130 is adjusted during that patient's inspiratory phase, with the position of the valve 130 determining the amount of resistance provided thereby. According to the illustrated example, the first valve 130 is in a partially open position during stage B, and the second valve 130 is in the fully open position during stage D, resulting in different pressures of air being delivered to the first patient and the second patient. In practice, it may be beneficial to be able to provide large differences in pressure delivery to patients being co-ventilated using the ventilator device 100 in order to accommodate the changing ventilation needs of the patients. According to various example uses, the ventilator device 100 may be operated in the pressure control ventilation mode to facilitate temporally split breaths with different and similar pressures and tidal volumes to allow for co-ventilation of multiple patients, independent of their lung compliance.

In some embodiments, the ventilator device 100 may be configured to operate in a volume control ventilation mode, according to which the controller 150 controls the actuators 140 to adjust the valves 130 to regulate the volume of air delivered to the respective patients based on settings input by the clinician for the different patients. Specifically, the clinician may use the user interface 160 to input a set of tidal volume settings for the patients, including a particular tidal volume setting for each of the respective patients, as well as a tidal volume setting of the ventilator 190 (i.e., the volume of air delivered from the ventilator 190 to the ventilator device 100 during a single inspiratory phase of the ventilator). The controller 150 may receive the set of tidal volume settings for the patients and the tidal volume setting of the ventilator 190 from the user interface 160 and may independently control the actuators 140) to adjust the valves 130 based at least in part on the set of tidal volume settings for the patients and the tidal volume setting of the ventilator 190. In particular, the controller 150 may control the actuators 140 to adjust the valves 130 such that the volumes of air that flow through the respective valves 130 correspond to the respective tidal volume settings for the respective patients. In this manner, each of the patients may receive customized breaths, despite the patients having different ventilation requirements. During operation of the ventilator device 100, the volume of air being provided by a particular one of the valves 130 may be changed by the clinician adjusting the tidal volume setting for the respective patient via the user interface 160. In this manner, the ventilator device 100 may provide dynamic control of the volumes of air being delivered to the patients, allowing the ventilator device 100 to adapt to changing ventilation needs of each patient as that patient's condition worsens or improves, without impacting the ventilator device's ability to provide adequate ventilation to the remaining patients.

As discussed above, in some embodiments, the ventilator device 100 may include flow sensors 184 in operable communication with the controller 150, with each of the flow sensors 184 being configured to detect a flow rate of air flow downstream of a respective one of the valves 130. The controller 150 may be configured to receive flow rate signals indicative of flow rates detected by the flow sensors 184 and to independently control the actuators 140 to adjust the valves 130 based at least in part on the flow rate signals. In this manner, one of the flow sensors 184 may be used to detect the flow rate of air being delivered to one of the patients during that patient's inspiratory phase, and the controller 150 may control the respective actuator 140 to adjust the respective valve 130 based on the flow rate detected by the flow sensor 184 and the tidal volume setting for the patient. For example, the controller may use the flow rate detected by the flow sensor 184 to calculate the volume of air delivered to the patient and then control the respective actuator 140 to adjust the respective valve 130 to the closed position when the calculated volume of air is equal to the patient's tidal volume setting. In this manner, the controller and the respective flow sensor 184 may implement a near real-time feedback loop for controlling the volume of air being delivered to the respective patient. Still other approaches for operating the ventilator device 100 in the volume control ventilation mode may be used in other embodiments.

According to one example of the ventilator device 100 operating in the volume control ventilation mode for co-ventilation of two patients, the tidal volume setting of the ventilator 190 may be 600 mL, the tidal volume setting for the first patient may be 400 mL. and the tidal volume for the second patient may be 200 mL. The inspiratory phase of the ventilator 190 may include two stages, a first stage corresponding to the inspiratory phase of the first patient and a second stage corresponding to the inspiratory phase of the second patient. During the first stage, the first valve 130 (i.e., the valve 130 corresponding to the first patient) of the ventilator device 100 may be in the open position or a partially opened position while the second valve 130 (i.e., the valve 130 corresponding to the second patient) is in the closed position, such that a tidal volume of 400 mL of air is delivered from the ventilator device 100 to the first patient, while no air is delivered from the ventilator device 100 to the second patient. During the second stage, the second valve 130 may be in the open position or a partially opened position while the first valve 130 is in the closed position, such that a tidal volume of 200 mL of air is delivered from the ventilator device 100 to the first patient, while no air is delivered from the ventilator device 100 to the second patient. During the expiratory phase of the ventilator 190, both of the valves 130 may be in the closed position, such that no air is delivered from the ventilator device 100 to either of the patients. According to various example uses, the ventilator device 100 may be operated in the volume control ventilation mode to facilitate temporally split breaths with different and similar pressures and tidal volumes to allow for co-ventilation of multiple patients, independent of their lung compliance.

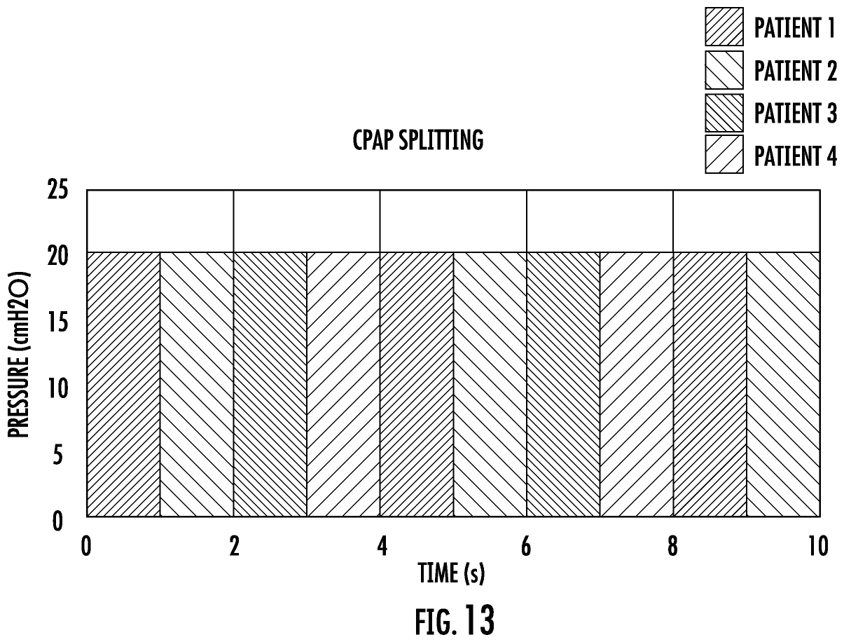

In some embodiments, the ventilator 190 may be operated in a continuous positive airway pressure (CPAP) mode, such that the ventilator 190 provides continuous airflow output at a predetermined pressure, and the ventilator device 100 may be used to distribute portions of the airflow to a plurality of patients. By receiving a continuous flow of air from the ventilator 190, the ventilator device 100 may provide co-ventilation for an increased number of patients. FIG. 13 illustrates an example ventilation schedule for the ventilator device 100 being used with the ventilator 190 operating in the CPAP mode for co-ventilation of four patients. As shown, the ventilator device 100 may proceed through a set of four stages each corresponding to one of the patients (i.e., a first stage for a first patient, a second stage for a second patient, a third stage for a third patient, and a fourth stage for a fourth patient) in a repeating manner, with each stage having a duration of one second. During the first stage, a first valve 130 of the ventilator device 100 corresponding to the first patient may be in the open position or a partially open position while the remaining valves 130 are in the closed position, such that air is delivered from the ventilator device 100 to the first patient, while no air is delivered from the ventilator device 100 to the second, third, or fourth patients. During the second stage, a second valve 130 of the ventilator device 100 corresponding to the second patient may be in the open position or a partially open position while the remaining valves 130 are in the closed position, such that air is delivered from the ventilator device 100 to the second patient, while no air is delivered from the ventilator device 100 to the first, third, or fourth patients. During the third stage, a third valve 130 of the ventilator device 100 corresponding to the third patient may be in the open position or a partially open position while the remaining valves 130 are in the closed position, such that air is delivered from the ventilator device 100 to the third patient, while no air is delivered from the ventilator device 100 to the first, second, or fourth patients. During the fourth stage, a fourth valve 130 of the ventilator device 100 corresponding to the fourth patient may be in the open position or a partially open position while the remaining valves 130 are in the closed position, such that air is delivered from the ventilator device 100 to the fourth patient, while no air is delivered from the ventilator device 100 to the first, second, or third patients. According to the illustrated example, air may be delivered from the ventilator device 100 to each of the patients at the same pressure. In other embodiments, air may be delivered from the ventilator device 100 to the respective patients at different pressures by the controller 150 controlling the respective actuators 140 to adjust the respective valves 130 to positions corresponding to a set of inspiratory pressure settings for the patients, as discussed above.

In some embodiments, the time-multiplexing approach and the electromechanical control of the actuators 140 of the ventilator device 100 may be combined with the ventilator 190 being operated in a CPAP mode to ventilate multiple patients with different I:E ratios for the patients. When operating in the CPAP mode, the ventilator 190 may be constantly sensing and waiting for a decrease in pressure in the ventilation circuit to deliver air and raise the pressure back up to the set point. By individually controlling the actuators 140 to adjust the valves 130 between the open position and the closed position, each patient's branch may be opened in rhythm to a separate I:E ratio, and the ventilator 190 may produce variable flow rates to deliver air as appropriate for each of the patients. This approach may not allow for ventilation at different times for every breath, but resistances still may be induced to personalize the inspiratory pressure of the air delivered to each patient. Inspiratory times can be adjusted to allow variable inspiratory times and even reverse I:E ratios (i.e., where the inspiratory time is longer than the expiratory time, e.g., a I:E ratio of 1.5:1). In one example in which the ventilator device 100 is used to provide ventilation to two patients, a I:E ratio of 1:3 may be used for a first patient, and a I:E ratio of 1:1 may be used for a second patient. One implementation of this example is provided in Table 1, showing positions of the valves 130 for the respective patients at different times of a cycle. In another example in which the ventilator device 100 is used to provide ventilation to two patients, a I:E ratio of 1:3 may be used for a first patient, and a I:E ratio of 1.5:1 may be used for a second patient. One implementation of this example is provided in Table 2, showing positions of the valves 130 for the respective patients at different times of a cycle.

TABLE 1

| | Time (sec) | | | | | | | | |
|---|---|---|---|---|---|---|---|---|---|
| | 0 | 0.5 | 1 | 1.5 | 2 | 2.5 | 3 | 3.5 | 4 |
| Patient 1 | Closed | Closed | Closed | Closed | Open | Open | Closed | Closed | Closed |
| Valve Position | | | | | fully | fully | | | |
| Patient 2 | Open | Open | Closed | Closed | Open | Open | Closed | Closed | Open |
| Valve Position | 70% | 70% | | | 70% | 70% | | | 70% |

| | Time (sec) | | | | | | | |
|---|---|---|---|---|---|---|---|---|
| | 4.5 | 5 | 5.5 | 6 | 6.5 | 7 | 7.5 | 8 |
| Patient 1 | Closed | Closed | Closed | Open | Open | Closed | Closed | Closed |
| Valve Position | | | | fully | fully | | | |
| Patient 2 | Open | Closed | Closed | Open | Open | Closed | Closed | Open |
| Valve Position | 70% | | | 70% | 70% | | | 70% |

TABLE 2

| | Time (sec) | | | | | | | | |
|---|---|---|---|---|---|---|---|---|---|
| | 0 | 0.5 | 1 | 1.5 | 2 | 2.5 | 3 | 3.5 | 4 |
| Patient 1 | Closed | Closed | Closed | Closed | Open | Open | Closed | Closed | Closed |
| Valve Position | | | | | fully | fully | | | |
| Patient 2 | Open | Open | Open | Closed | Closed | Open | Open | Open | Closed |
| Valve Position | 70% | 70% | 70% | | | 70% | 70% | 70% | |

| | Time (sec) | | | | | | | |
|---|---|---|---|---|---|---|---|---|
| | 4.5 | 5 | 5.5 | 6 | 6.5 | 7 | 7.5 | 8 |
| Patient 1 | Closed | Closed | Closed | Open | Open | Closed | Closed | Closed |
| Valve Position | | | | fully | fully | | | |
| Patient 2 | Closed | Open | Open | Open | Closed | Closed | Open | Open |
| Valve Position | | 70% | 70% | 70% | | | 70% | 70% |

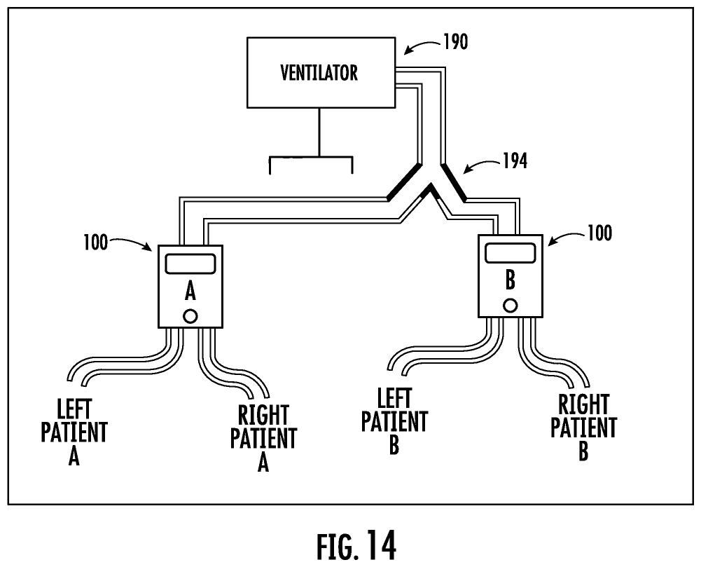

In some embodiments, multiple of the ventilator devices 100 may be used with a single ventilator 190 for co-ventilation of a plurality of patients. For example, FIG. 14 illustrates a pair of the ventilator devices 100 being used with a single ventilator 190 for co-ventilation of four patients. As shown, an upstream air tube splitter 194 may be provided to split airflow from the ventilator 190 into two streams of air for the respective ventilator devices 100. In this manner, one ventilator tubing line may extend from inspiratory port of the ventilator 190 to an inlet of the upstream air tube splitter 194, another ventilator tubing line may extend from one outlet of the upstream air tube splitter 194 to the inlet 112 of the ventilator circuit 110 of the first ventilator device 100, and another ventilator tubing line may extend from another outlet of the upstream air tube splitter 194 to the inlet 112 of the ventilator circuit of the second ventilator device 100. As shown, each of the ventilator devices 100 may be used to ventilate two of the four patients. The ventilator devices 100 may be operated in a manner similar to that described above for pressure control ventilation or volume control ventilation. In some embodiments, the inspiratory pressure and/or the flow rate of the ventilator 190 may be doubled, as compared to above examples using a single ventilator device 100, in order to provide sufficient air for ventilating the four patients using the pair of ventilator devices 100. Various other arrangements of multiple ventilator devices 100 and a single ventilator 190 may be used for co-ventilating a plurality of patients in other embodiments.

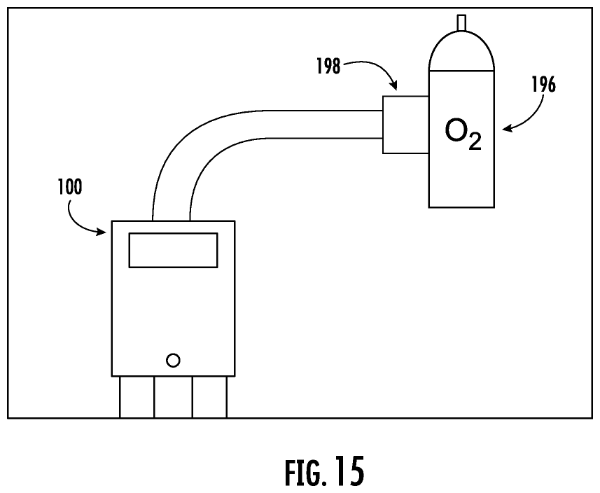

In some embodiments, the ventilator device 100 may be used with a source of pressurized air other than a ventilator for ventilating a plurality of patients. For example, FIG. 15 illustrates the ventilator device 100 being used with an oxygen tank 196 for co-ventilation of multiple patients. As shown, a ventilator tubing line may extend between the oxygen tank 196 and the inlet 112 of the ventilator circuit 110 of the ventilator device 100. In some embodiments, the oxygen tank 196 may provide continuous airflow output at a predetermined pressure and/or flow rate, and the ventilator device 100 may be used to distribute portions of the airflow to a plurality of patients. In some embodiments, as shown, a valve 198 may be provided between the oxygen tank 196 and the ventilator device 100, such as at an outlet of the oxygen tank 196. The valve 198 may be configured to open and close in a manner similar to a ventilator to control delivery of air from the oxygen tank 196 to the ventilator device 100. In some embodiments, the timing of the opened and closed states of the valve 198 may correspond to the opened and closed states of the valves 130 of the ventilator device 100 to allow for synchronization. The ventilator device 100 may be operated in a manner similar to that described above for pressure control ventilation of volume control ventilation. In some instances, the ventilator device 100 may be used with the oxygen tank 196 in in situations in which a ventilator is not available. Still other types of air supplies may be used with the ventilator device 100 in other embodiments.

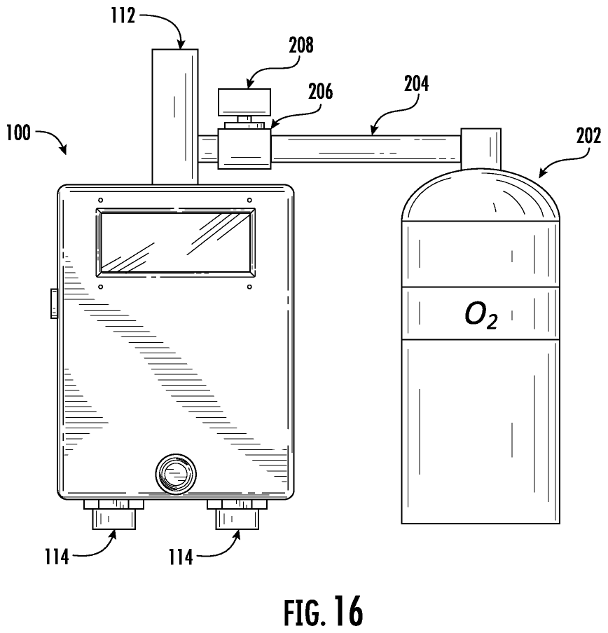

In some embodiments, the ventilator device 100 may be used with a source of pressurized air and a source of oxygen in ventilating a plurality of patients, with the ventilator device 100 being configured to control a fraction of inspired oxygen (FiO₂) for each of the patients. For example, FIG. 16 illustrates the ventilator device 100 being used with an oxygen tank 202 that provides a flow of oxygen to the ventilator device 100 via a conduit 204. As shown, a valve 206 may be coupled to the conduit 204 and configured to be adjusted to regulate the flow of oxygen into the ventilator circuit 110 of the ventilator device 100, and an actuator 208 may be coupled to the valve 206 and configured to adjust the valve 206. The controller 150 may be in operable communication with the actuator 208 and configured to control the actuator 208 to adjust the valve 206 such that a desired amount of oxygen is delivered into the ventilator circuit 110 for the respective patients. In this manner, while employing temporal multiplexing, as discussed above, the ventilator device 100 may provide customized FiO₂ levels for the respective patients. Particular FiO₂ level settings for the respective patients may be input by the clinician using the user interface 160. In some embodiments, the sensors 180 of the ventilator device 100 may include an oxygen flow meter configured to detect a flow rate of oxygen being delivered into the ventilator circuit 110, and the controller 150 may cause the actuator 208 to adjust the valve 206 to a particular position based at least in part on the detected flow rate of oxygen.

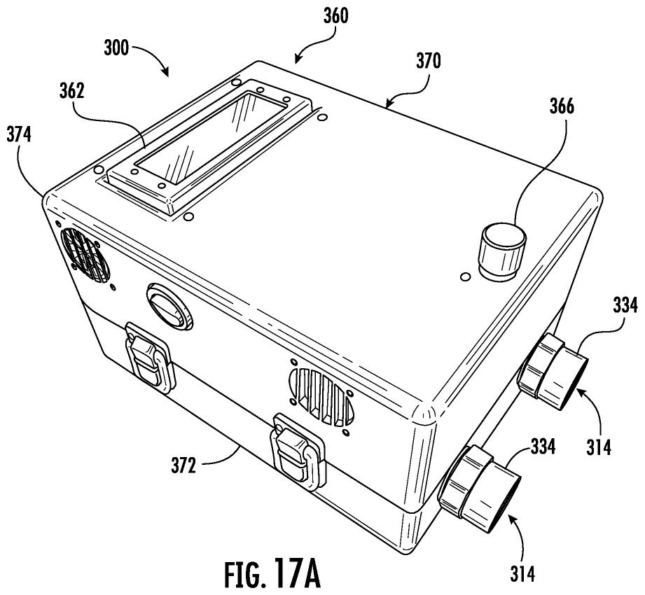

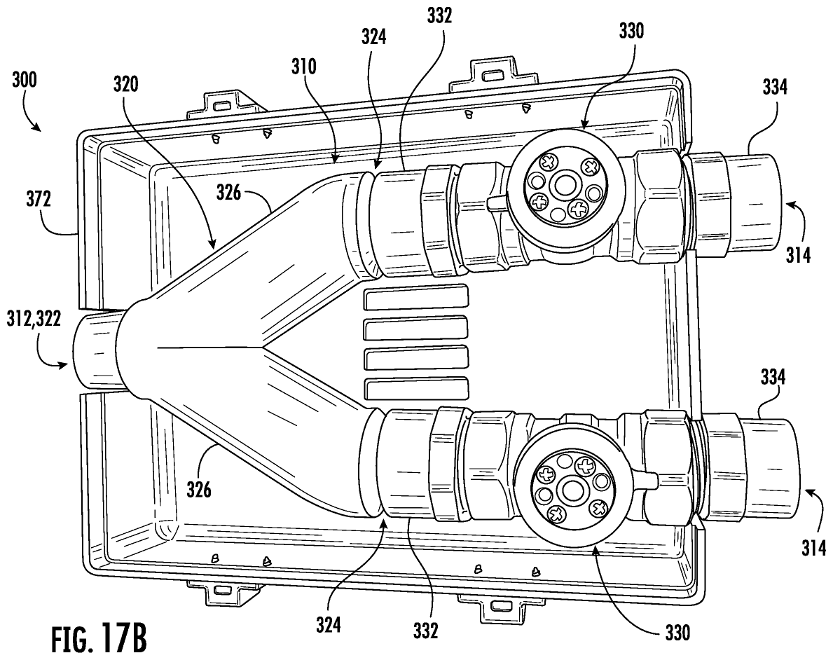

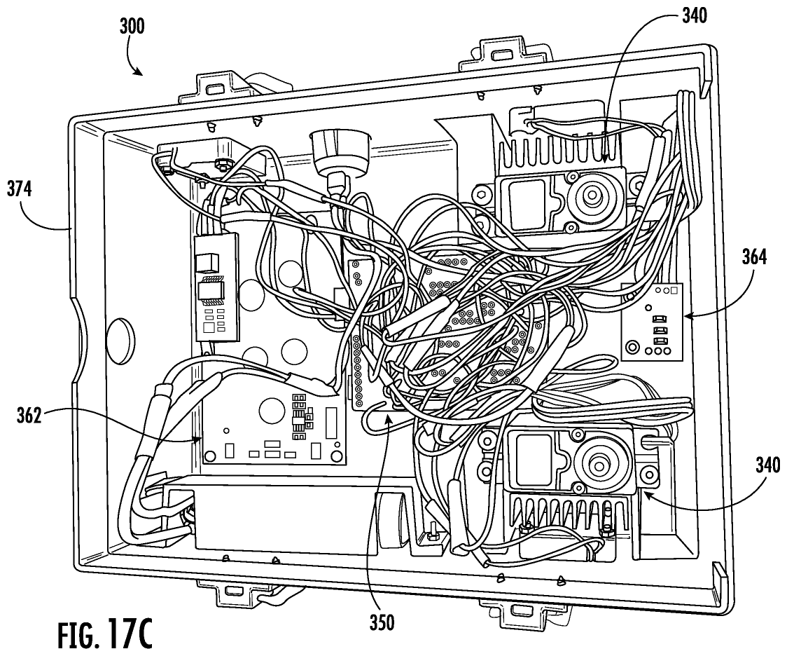

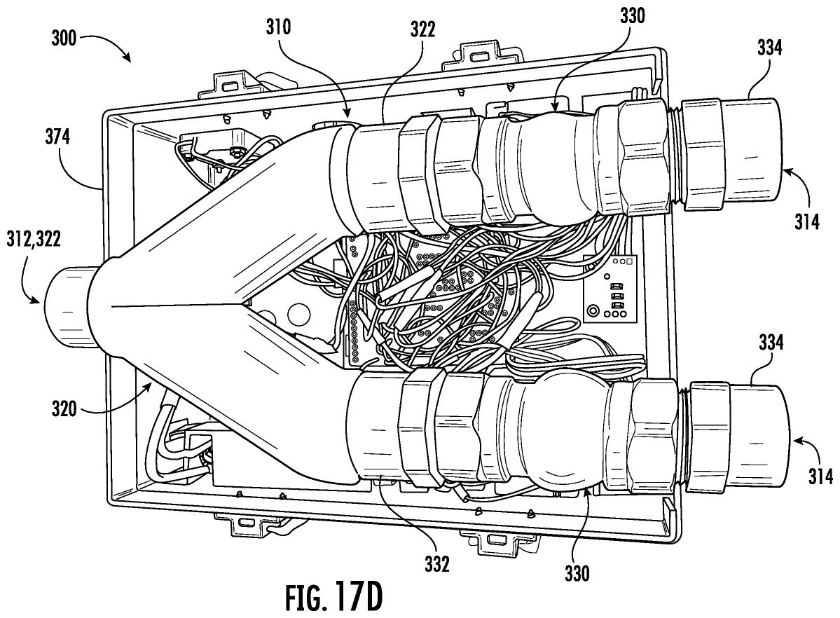

FIGS. 17A-17D depict another example ventilator device 300 and components thereof in accordance with embodiments of the disclosure, which may be used with a ventilator 190 or other source of pressurized air to provide co-ventilation of multiple patients, as described herein. The ventilator device 300 generally may be configured in a manner similar to the ventilator device 100 described above. Certain similarities and differences between the ventilator device 300 and its components and the ventilator device 100 and its components will be appreciated from the figures.

As shown in FIGS. 17A-17D, the ventilator device 300 generally may include a ventilator circuit 310 having a single inlet 312 and a plurality of outlets 314. During use of the ventilator device 300, the inlet 312 may be connected to the ventilator 390 or other source of pressurized air by a standard ventilator tubing line for receiving air therefrom, and the outlets 314 may be connected to respective patients by additional ventilator tubing lines for delivering air to the patients. According to the illustrated example, the ventilator circuit 310 may include an air tube splitter 320, a plurality of valves 330, a plurality of first connectors 332, and a plurality of second connectors 334. The air tube splitter 320 may be configured to divide an incoming flow of air from the ventilator 390 into separate streams, and the valves 330 may be configured to regulate the respective streams of air flowing through the ventilator circuit 310 and ultimately to the respective patients being ventilated. The ventilator device 300 also may include a plurality of actuators 340 and a controller 350 in operable communication with the actuators 340. The actuators 340 may be coupled to the valves 330, with each of the actuators 340 being configured to adjust a respective one of the valves 330. The controller 350 may be configured to receive certain settings, such as a set of inspiratory pressure settings or tidal volume settings for the patients, and to independently control the actuators 340 to adjust the valves 330 based at least in part on the settings, such as the set of inspiratory pressure settings or tidal volume settings. The ventilator device 300 also may include a user interface 360 in operable communication with the controller 350. The user interface 360 may be configured to allow a user, such as a clinician, to input and adjust the set of inspiratory pressure settings or tidal volume settings for the patients as well as other settings or operating parameters, as described below. The user interface 360 also may be configured to allow the user to monitor various settings and operating parameters during use of the ventilator device 300. As shown, the ventilator device 300 also may include an enclosure 370 that houses other components of the device 300, such as the valves 330, the actuators 340, the controller 350, and at least part of the air tube splitter 320, therein.

As shown in FIG. 17B, the air tube splitter 320 may include a single inlet 322, a plurality of outlets 324 in fluid communication with the inlet 322, and a plurality of branches 326, with each of the branches 326 extending to a respective one of the outlets 324. In this manner, the air tube splitter 320 may be configured to channel air received from the ventilator 390 and distribute the air among a plurality of ventilation paths to provide co-ventilation of a plurality of patients. In some embodiments, as shown, the inlet 322 of the air tube splitter 320 may define the inlet 312 of the overall ventilator circuit 310. As shown, the branches 326 of the air tube splitter 320 may be disposed at an angle with respect to one another, which may be selected to provide desired flow characteristics of air passing through the air tube splitter 320 and the respective branches 326 thereof.

As shown in FIG. 17B, the valves 330 may be configured to regulate air flow through the ventilator circuit 300 to the outlets 314 thereof and ultimately to the plurality of patients being ventilated using the ventilator device 300. In particular, the valves 330 may be used to regulate air flow and provide flow characteristics (e.g., pressure, flow rate, etc.) at each of the outlets 314 in a manner that meets inspiratory pressure requirements and/or tidal volume requirements of the respective patients connected to the ventilator device 300. As shown, each of the valves 330 may be coupled to a respective one of the branches 326 and configured to be adjusted to regulate air flow through the respective branch 326 to the respective outlet 314. Each of the valves 330 may be configured to be adjusted between an open position (i.e., a fully open position) and a closed position (i.e., a fully closed position). When in the open position, the valve 330 may allow air to freely pass through the respective branch 326 to the respective outlet 314. When in the closed position, the valve 340 may prevent air from passing through the respective branch 326 to the respective outlet 314. In some embodiments, as shown, each of the valves 330 also may be configured to be adjusted to a plurality of partially open positions between the open position and the closed position. In this manner, during use of the ventilator device 300, each of the valves 330 may adjusted to a position suitable for providing desired flow characteristics of air delivered to a respective patient, as described further below. In other words, each of the valves 330 may be adjusted to provide a varying resistance to air flow through a respective portion of the ventilator circuit 310 to a respective outlet 314 thereof. In some embodiments, as shown, each of the valves 330 may be a ball valve, although other types of valves may be used in other embodiments.

In some embodiments, the valves 330 may be coupled to the branches 326 of the air tube splitter 320 by the first connectors 332. As shown, the first connectors 332 may be coupled to the branches 326 and the valves 230, with each of the first connectors 232 connecting a respective one of the valves 330 to a respective one of the branches 326. In some embodiments, the second connectors 334 may be coupled to the valves 330 for connecting respective ventilation tubing lines extending between the ventilator device 300 and the patients being ventilated. As shown, the second connectors 334 may be coupled to the valves 330, with each of the second connectors 334 being configured to connect a ventilation tubing line to a respective one of the valves 330. In some embodiments, as shown, the second connectors 334 may define the outlets 314 of the overall ventilator circuit 310.

The actuators 340 may be coupled to the valves 330 and configured to adjust the positions of the respective valves 330 to regulate air flow through the ventilator circuit 300 in a desired manner for ventilating the patients connected to the ventilator device 300. In particular, each of the actuators 340) may be configured to adjust a respective one of the valves 330 between the open position, closed position, and partially open positions of the valve 330. The actuators 340 may be controlled by the controller 350 to adjust the valves 330 in a desired manner. In some embodiments, each of the actuators 340) may be a motor, such as a servo motor, as shown, although other types of actuation devices may be used in other embodiments.

The controller 350 may be in operable communication with the actuators 340 as well as other electronic components of the ventilator device 300. As shown, the controller 350 may be in operable communication with the actuators 340 and the user interface 360. In some embodiments, the controller 350 may be in operable communication with one or more sensors of the ventilator device 300, which may be similar to the sensors 180 discussed above. The controller 350 may be configured to control operation of the actuators 340 to adjust the valves 330 based at least in part on one or more settings and/or one or more operating parameters of the ventilator device 300. In some embodiments, the controller 350 may be configured to receive one or more settings for the patients being treated using the ventilator device 300 and to independently control the actuators 340 to adjust the valves 330 based at least in part on the settings. According to various embodiments, the controller 350 may be configured to interact with and control the other components of the ventilator device 300 in a manner similar to the controller 150 described above with respect to the ventilator device 100. In other words, the controller 350 may provide the same functionality as the controller 150 described above.

As shown in FIGS. 17A-17D, the user interface 360 may include a display screen 362, a rotary encoder 364, and a knob 366 coupled to the rotary encoder 364. The display screen 362 may be configured to display the one or more settings and/or one or more operating parameters of the ventilator device 300 and/or the ventilator 190 discussed above. In some embodiments, the display screen 362 may be a liquid-crystal display (LCD) screen, although other suitable types of display screens may be used in other embodiments. The knob 366 may be configured to be rotated, depressed, and/or otherwise manipulated by a user to allow the user to input and adjust the one or more settings and/or one or more operating parameters via the rotary encoder 364. Various other configurations of the user interface 360 may be used in different embodiments.

The electronic components of the ventilator device 300, including the actuators 340, the controller 350, the user interface 360, and the sensors 380, may be powered by an external power source. For example, the ventilator device 300 may be connected to mains power for providing power to the electronic components. In some embodiments, as shown, the ventilator device 300 also may include an internal power source, such as one or more batteries or other power storage devices, as a backup to the external power source. In this manner, the internal power source may allow for temporary use of the ventilator device 300 while external power is not available, for example, during intermittent power outages. Because the ventilator device 300 is intended for use in emergency situations, the internal power source may be particularly beneficial for patient safety, enabling continuous operation of the ventilator device 300.

As shown in FIGS. 17A-17D, the enclosure 370 may be configured to house other components of the ventilator device 300 therein. The enclosure 370 may protect the enclosed components and allow the ventilator device 300 to be safely and conveniently transported and installed in various applications. According to the illustrated example, the enclosure 370 may house the valves 330, the actuators 340, the controller 350, and at least part of the air tube splitter 320 therein. As shown, the inlet 322 of the air tube splitter 320 may protrude from one end of the enclosure 370 for connection to the ventilator 190 via a ventilation tubing line, and the second connectors 334 may protrude from the other end of the enclosure 370 for connection to the patients via additional ventilation tubing lines. In some embodiments, as shown, the enclosure 370 may include a first portion 372 and a second portion 374 that are removably coupled to one another. The first portion 372 and the second portion 374 may be coupled to one another during use of the ventilator device 300 and may be decoupled to allow the enclosure 370 to be opened, for example, to facilitate maintenance or cleaning of the internal components. Various types of mechanical fasteners may be used to removably couple the first portion 372 and the second portion 374 to one another. In some embodiments, as shown, the first portion 372 may house at least part of the air tube splitter 320, the valves 330, and the first connectors 332 therein, and the second portion 374 may house at least part of the actuators 340, the controller 350, the display screen 362, and the rotary encoder 364 therein. Various other configurations of the enclosure 370 may be used in other embodiments.

The ventilator devices 100, 300 described herein may be used in various types of environments for safely co-ventilating various types of patients, as needed, when ventilator demand exceeds availability. In some instances, the ventilator devices 100, 300 may be used in the event of pandemics, such as the COVID-19 pandemic, chemical warfare, environmental disasters, mass shootings, or other locoregional disturbances that may cause increased demand. The ventilator devices 100, 300 may be beneficial in developing countries or areas with limited ventilator resources. The ventilator devices 100, 300 also may be useful for the military, particularly in remote areas, where appropriate ventilator supply may be limited or logistically difficult because of travel needs. In various instances, the ventilator devices 100, 300 may be used for multiple adult or pediatric patients in hospitals or other types of healthcare facilities. In some instances, the ventilator devices 100, 300 may be used in neonatal applications, to provide ventilation to multiple infants. The ventilator devices 100, 300 also may be used for co-ventilation of multiple animals. Comparative medicine and veterinary practices often use ventilators for pre-clinical studies or life-saving procedures. Use of the ventilator devices 100, 300 employing the time-multiplexing approach for animal surgeries or life support may increase the quality of life for the animals.

The ventilator devices 100, 300 often may be used in hospitals or other types of healthcare facilities having ventilators. In some instances, the ventilator devices 100, 300 may be moved from one location to another in such facilities and then supported by existing structure in the area of the patients being treated. For example, the ventilator devices 100, 300 may be temporarily mounted to or otherwise supported by a ventilator with which the devices 100, 300 are being used, for example, via a hook or other type of fastener provided with the devices 100, 300. In some instances, the ventilator devices 100, 300 may be permanently or temporarily mounted on a wall in a hospital or other healthcare facility. In hospitals, air and oxygen gas lines often are embedded into the walls of the hospital. In such instances, the ventilator devices 100, 300 may be mounted to the same walls, mediating the gas lines to patients and providing the ability to safely ventilate multiple patients in a time multiplexed manner to increase hospital capacity. In some instances, the ventilator devices 100, 300 may be provided within emergency vehicles, such as ambulances. Emergency vehicles often require the ability to mechanically ventilate a patient. During mass casualty events, an ambulance carrying a single patient can cause delays to others who need similar life-saving procedures, and thus embedding the ventilator devices 100, 300 into emergency vehicles may increase the number of patients that can be safely transported to a hospital for further care. As will be appreciated, the ventilator devices 100, 300 may be used in various types of environments other than traditional healthcare facilities.

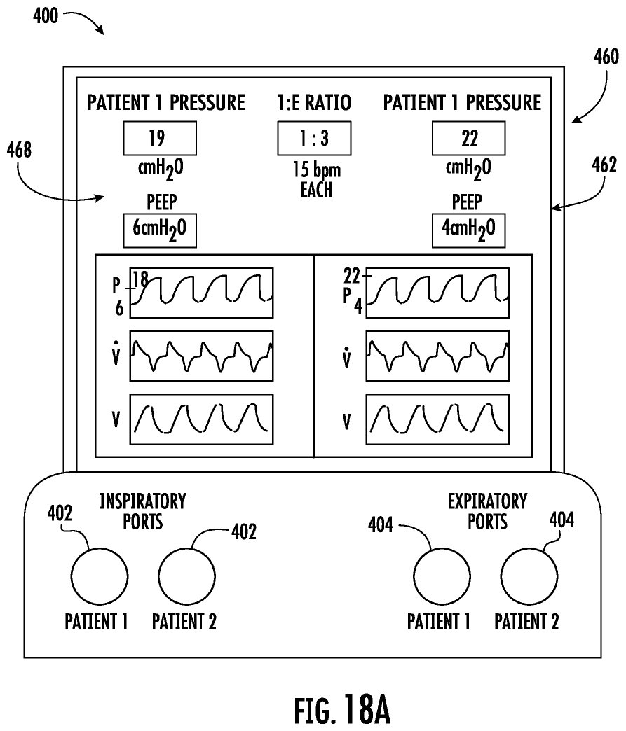

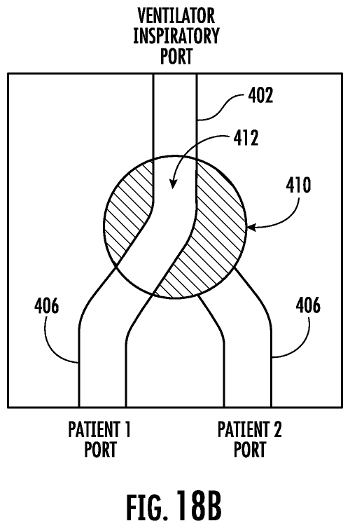

In some embodiments, the functionality of the ventilator devices 100, 300 described above may be incorporated into a ventilator. In other words, instead a ventilator being used along with one of the ventilator devices 100, 300, a ventilator itself may be configured to provide the functionality of the ventilator devices 100, 300 to allow safe co-ventilation of multiple patients. FIG. 18A depicts an example ventilator 400 in accordance with embodiments of the disclosure, which may be used to provide co-ventilation of multiple patients. As shown, the ventilator 400 may include a plurality of inspiratory ports 402 for delivering air to the patients via respective ventilator tubing lines, and a plurality of expiratory ports 404 for receiving air from the patients via respective ventilator tubing lines. The ventilator 400 may include various components similar to the components of the ventilator devices 100, 300 described above for carrying out similar functions. The ventilator 400 may employ temporal multiplexing, as discussed above, for delivering air to the patients in accordance with settings input by the clinician.

As shown in FIG. 18A, the ventilator 400 may include a user interface 460 may be configured to allow the clinician to input and adjust one or more settings and/or one or more operating parameters of the ventilator 400. For example, the user interface 460 may be configured to allow a user to input and adjust one or more settings for the patients being ventilated using the ventilator 400, such as a set of inspiratory pressure settings or tidal volume settings for the patients, an inspiratory to expiratory ratio for the patients, and/or other settings for the patients. The user interface 460 also may allow a user to input and adjust one or more settings of the ventilator 400, such as an inspiratory pressure setting of the ventilator 400, an inspiratory to expiratory ratio of the ventilator 400, and/or other settings of the ventilator 400. In some embodiments, the user interface 460 may be configured to display the one or more settings for the patients and the one or more settings and/or one or more operating parameters of the ventilator 400, such that a user may monitor the settings and/or operating parameters. As shown, the user interface 460 may include a display screen 462 configured to display the one or more settings for the patients and the one or more settings and/or one or more operating parameters of the ventilator 400. In some embodiments, the display screen 462 may be a touchscreen display. In this manner, a user may interact directly with the display screen 462 to adjust settings and/or operating parameters, obviating the need for a knob or other input device. The display screen 462 may provide a GUI 468 that displays various settings and operating parameters of the ventilator 400 for the user. As shown, the GUI 468 may display independent patient data for each of the patients being treated using the ventilator 400.

In some embodiments, the ventilator 400 may have only a single inspiratory port 402 but may include a valve assembly for controlling the flow of air from the inspiratory port 402 to multiple patients. FIG. 18B depicts an example a valve assembly 410 as may be used for the ventilator 400. As shown, the valve assembly 410 may be disposed between the inspiratory port 402 and a plurality of patient ports 406, each of which may be connected to a respective patient via a ventilator tubing line. The valve assembly 410 may be configured to selectively connect one of the patient ports 406 to the inspiratory port 402. As shown, the valve assembly 410 may include a rotating valve having a passage 412 extending therethrough. The valve may be configured to rotate between a first position in which the passage 412 connects one of the patient ports 406 to the inspiratory port 402 and a second position in which the passage 412 connects the other of the patient ports 406 to the inspiratory port 402. Various other configurations of the valve assembly 410 may be used in other embodiments for switching the flow of air from the inspiratory port 402 to one of the patient ports 406. In some embodiments, the valve assembly 410 may be adjusted by an actuator, such as a motor, coupled to the valve assembly 410, and the controller of the ventilator 400 may be in operable communication with the actuator and configured to control the actuator to adjust the valve assembly 410 at appropriate times for co-ventilation of the patients. In some embodiments, the ventilator 400 may have a pre-programmed co-ventilation mode for dynamically delivering variable breaths to a plurality of patients in a time-multiplexed manner, as discussed above.

A number of example embodiments are provided herein. However, it is understood that various modifications can be made without departing from the spirit and scope of the disclosure herein. As used in the specification, and in the appended claims, the singular forms “a.” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed, while specific reference of each various individual and collective combinations and permutations of these components may not be explicitly disclosed, each is specifically contemplated and described herein. For example, if a device is disclosed and discussed each and every combination and permutation of the device, and the modifications that are possible are specifically contemplated unless specifically indicated to the contrary. Likewise, any subset or combination of these is also specifically contemplated and disclosed. This concept applies to all aspects of this disclosure including. but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.

1. A ventilator device for co-ventilation of multiple patients, the ventilator device comprising:
an air tube splitter having an inlet, a plurality of outlets, and a plurality of branches, wherein each of the branches extends to a respective one of the outlets;
a plurality of valves coupled to the branches, wherein each of the valves is configured to be adjusted between an open position and a closed position, wherein each of the valves is configured to be adjusted to regulate air flow through a respective one of the branches;
a plurality of actuators coupled to the valves, wherein each of the actuators is configured to adjust a respective one of the valves; and
a controller in operable communication with the actuators, wherein the controller is configured to:
receive a set of inspiratory pressure settings or tidal volume settings for the patients; and
independently control the actuators to adjust the valves based at least in part on the set of inspiratory pressure settings or tidal volume settings.

2. (canceled)

3. (canceled)

4. The ventilator device of claim 1, wherein the plurality of branches comprises a first branch and a second branch disposed adjacent to one another and defining an angle therebetween.

5. The ventilator device of claim 4, wherein the angle is fixed.

6. (canceled)

7. The ventilator device of claim 4, wherein the angle is adjustable between 15 degrees and 180 degrees.

8. (canceled)

9. (canceled)

10. (canceled)

11. (canceled)

12. (canceled)

13. The ventilator device of claim 1, wherein the controller is further configured to control the actuators to alternately adjust the valves between the open position and the closed position based at least in part on a predetermined inspiratory to expiratory ratio.

15. (canceled)

16. The ventilator device of claim 1, wherein the controller is further configured to control the actuators to adjust the valves such that:
a first valve and a second valve of the plurality of valves are in the closed position for a first time period;
the first valve is in the open position or a partially open position while the second valve is in the closed position for a second time period following the first time period;
the second valve is in the open position or a partially open position while the first valve is in the closed position for a third time period following the second time period; and
the first valve and the second valve are in the closed position for a fourth time period following the third time period.

17. (canceled)

18. (canceled)

19. The ventilator device of claim 1, further comprising a plurality of first connectors coupled to the branches and the valves and a plurality of second connectors coupled to the valves, wherein each of the first connectors connects a respective one of the valves to a respective one of the branches and each of the second connectors is configured to connect a ventilation tubing line to a respective one of the valves.

20. (canceled)

21. The ventilator device of claim 19, wherein the air tube splitter, the first connectors, the valves, and the second connectors are separately formed and connected to one another.

22. The ventilator device of claim 19, wherein the air tube splitter, the first connectors, portions of the valves, and the second connectors are integrally formed with one another.

23. (canceled)

24. (canceled)

25. (canceled)

26. (canceled)

27. (canceled)

28. (canceled)

29. The ventilator device of claim 1, further comprising a user interface in operable communication with the controller, wherein the user interface is configured to allow a user to input and adjust the set of inspiratory pressure settings and/or tidal volume settings for the patients, wherein the user interface is further configured to allow the user to input and adjust an inspiratory to expiratory ratio for the patients.

30. (canceled)

31. The ventilator device of claim 29, wherein the user interface comprises a display screen, a rotary encoder, and a knob coupled to the rotary encoder.

32. The ventilator device of claim 29, wherein the user interface comprises a graphical user interface configured to display independent patient data for each of the patients, wherein the user interface comprises a touchscreen display.

33. (canceled)

34. (canceled)

35. (canceled)

36. The ventilator device of claim 1, further comprising a ventilator coupled to the inlet of the air tube splitter by a ventilation tubing line and configured to deliver air to the inlet of the air tube splitter, wherein the controller is in operable communication with the ventilator and configured to control an air output of the ventilator.

37. (canceled)

38. The ventilator device of claim 36, wherein the ventilator is configured to deliver air to the inlet of the air tube splitter at a respiratory rate that is at least double a single patient respiratory rate requirement.

39. The ventilator device of claim 36, wherein the ventilator is configured to deliver air to the inlet of the air tube splitter at a flow rate that is at least double a single patient respiratory rate requirement.

40. The ventilator device of claim 1, further comprising a continuous positive airway pressure device coupled to the inlet of the air tube splitter by a ventilation tubing line and configured to deliver air to the inlet of the air tube splitter.

41. The ventilator device of claim 1, further comprising an oxygen tank coupled to the inlet of the air tube splitter by a ventilation tubing line and configured to deliver air to the inlet of the air tube splitter.

42. The ventilator device of claim 41, wherein the oxygen tank is configured to deliver air to the inlet of the air tube splitter at a flow rate that is at least double a single patient respiratory rate requirement.

43. A ventilator system for co-ventilation of multiple patients, the ventilator system comprising:
a first ventilation device and a second ventilation device each comprising:
an air tube splitter having an inlet, a plurality of outlets, and a plurality of branches, wherein each of the branches extends to a respective one of the outlets;
a plurality of valves coupled to the branches, wherein each of the valves is configured to be adjusted to regulate air flow through a respective one of the branches;
a plurality of actuators coupled to the valves, wherein each of the actuators is configured to adjust a respective one of the valves; and
a controller in operable communication with the actuators, wherein the controller is configured to:
receive a set of inspiratory pressure settings or tidal volume settings for the patients; and
independently control the actuators to adjust the valves based at least in part on the set of inspiratory pressure settings or tidal volume settings; and
an upstream air tube splitter having an inlet and a plurality of outlets;
wherein the inlet of the air tube splitter of the first ventilation device is fluidically coupled to one of the outlets of the upstream air tube splitter, and wherein the inlet of the air tube splitter of the second ventilation device is fluidically coupled to another of the outlets of the upstream air tube splitter.

44. A method for co-ventilation of multiple patients, the method comprising:
delivering air from a ventilator to a ventilator device, the ventilator device comprising:
an air tube splitter having an inlet, a plurality of outlets, and a plurality of branches, wherein each of the branches extends to a respective one of the outlets;
a plurality of valves coupled to the branches, wherein each of the valves is configured to be adjusted to regulate air flow through a respective one of the branches;
a plurality of actuators coupled to the valves, wherein each of the actuators is configured to adjust a respective one of the valves; and
a controller in operable communication with the actuators;
receiving, via the controller, a set of inspiratory pressure settings or tidal volume settings for the patients; and
independently controlling, via the controller, the actuators to adjust the valves based at least in part on the set of inspiratory pressure settings or tidal volume settings.

45. (canceled)

46. (canceled)

47. (canceled)
